(19) United States
(12) Patent Application Publication
(10) Pub. No.: US 2023/0125707 A1
(43) Pub. Date: Apr. 27, 2023

(54) COMPOUND FOR ORGANIC OPTOELECTRONIC DEVICE, ORGANIC OPTOELECTRONIC DEVICE AND DISPLAY DEVICE

(71) Applicant: SAMSUNG SDI CO., LTD., Yongin-si (KR)

(72) Inventors: Mijin LEE, Suwon-si (KR); Jonghoon KIM, Suwon-si (KR); Youngmook LIM, Suwon-si (KR); Ho Kuk JUNG, Suwon-si (KR); Youngkyoung JO, Suwon-si (KR); Byoungkwan LEE, Suwon-si (KR); Sung-Hyun JUNG, Suwon-si (KR); Pyeongseok CHO, Suwon-si (KR); Jongwoo CHOI, Suwon-si (KR)

(21) Appl. No.: 17/901,977

(22) Filed: Sep. 2, 2022

(30) Foreign Application Priority Data
Sep. 3, 2021 (KR) 10-2021-0117782
Aug. 31, 2022 (KR) 10-2022-0110104

(51) Int. Cl. H01L 51/00; C07C 13/72

(52) U.S. Cl. CPC H01L 51/0058; C07C 13/72; H01L 51/5016

(57) ABSTRACT

A compound for an organic optoelectronic device, an organic optoelectronic device including the same, and a display device, the compound being represented by Chemical Formula I:

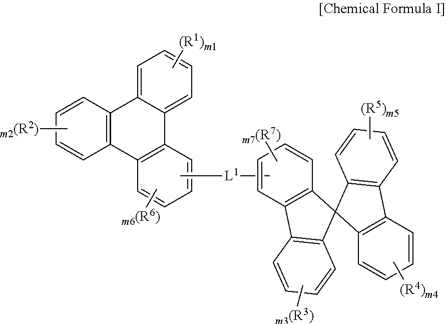

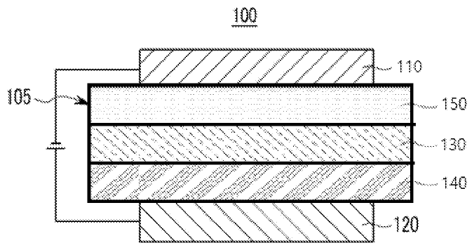

### CROSS-REFERENCE TO RELATED APPLICATION

This application claims priority to and the benefit of Korean Patent Application No. 10-2021-0117782 filed in the Korean Intellectual Property Office on Sep. 3, 2021, and Korean Patent Application No. 10-2022-0110104 filed in the Korean Intellectual Property Office on Aug. 31, 2022, the entire contents of which are incorporated herein by reference.

### BACKGROUND

### 1. Field

Embodiments relate to a compound for an organic optoelectronic device, an organic optoelectronic device, and a display device.

### 2. Description of the Related Art

An organic optoelectronic device (e.g., organic optoelectronic diode) is a device that converts electrical energy into photoenergy, and vice versa.

An organic optoelectronic device may be classified as follows in accordance with its driving principles. One is a photoelectric device where excitons generated by photoenergy are separated into electrons and holes and the electrons and holes are transferred to different electrodes respectively and electrical energy is generated, and the other is a light emitting device to generate photoenergy from electrical energy by supplying a voltage or a current to electrodes.

Examples of the organic optoelectronic device include an organic photoelectric device, an organic light emitting diode, an organic solar cell, and an organic photo conductor drum.

Among them, the organic light emitting diode (OLED) has recently drawn attention due to an increase in demand for flat panel displays. The organic light emitting diode converts electrical energy into light, and the performance of organic light emitting diode is greatly influenced by the organic materials disposed between electrodes.

### SUMMARY

The embodiments may be realized by providing a compound for an organic optoelectronic device, the compound being represented by Chemical Formula I:

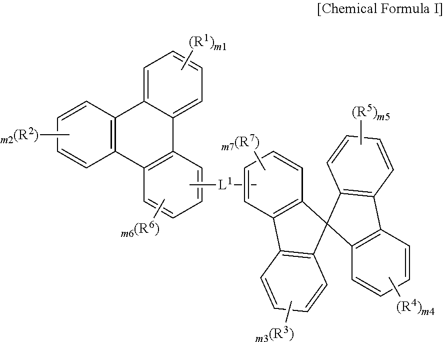

wherein, in Chemical Formula I, L¹ is a single bond, a substituted or unsubstituted C6 to C20 arylene group, a substituted or unsubstituted C2 to C20 heterocyclic group, or a combination thereof, R¹ to R⁷ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, m1 to m5 are each independently an integer of 1 to 4, and m6 and m7 are each independently an integer of 1 to 3, provided that the compound represented by Chemical Formula I satisfies at least one of the following conditions L¹ is a C6 to C20 arylene group substituted with at least one deuterium or a C2 to C20 heterocyclic group substituted with at least one deuterium; or at least one of R¹ to R⁷ is deuterium, a C6 to C30 aryl group substituted with at least one deuterium, or a C2 to C30 heterocyclic group substituted with at least one deuterium.

The embodiments may be realized by providing an organic optoelectronic device including an anode and a cathode facing each other, and at least one organic layer between the anode and the cathode, wherein the at least one organic layer includes the compound for an organic optoelectronic device according to an embodiment.

The embodiments may be realized by providing a display device including the organic optoelectronic device according to an embodiment.

### BRIEF DESCRIPTION OF THE DRAWING

Features will be apparent to those of skill in the art by describing in detail exemplary embodiments with reference to the attached drawings in which:

the FIGURE is a cross-sectional view illustrating an organic light emitting diode according to an embodiment.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURES, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout. As used herein, the term “or” is not an exclusive term, e.g., “A or B” would include A, B, or A and B.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In addition, in specific examples of the present invention, “substituted” refers to replacement of at least one hydrogen of a substituent or a compound by deuterium, a C1 to C20 alkyl group, a C6 to C30 aryl group, or a cyano group. In addition, in specific examples of the present invention, “substituted” refers to replacement of at least one hydrogen of a substituent or a compound by deuterium, a C1 to C5 alkyl group, a C6 to C18 aryl group, or a cyano group. In addition, in specific examples of the present invention, “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of a hydrogen atom by another substituent and remaining of the hydrogen atom.

As used herein, “hydrogen substitution (—H)” may include “deuterium substitution (-D)” or “tritium substitution (-T).”

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example, a fluorenyl group, and the like.

The aryl group may include a monocyclic, polycyclic or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to the lowest unoccupied molecular orbital (LUMO) level.

Hereinafter, a compound for an organic optoelectronic device according to an embodiment is described.

The compound for an organic optoelectronic device according to an embodiment may be represented by, e.g., Chemical Formula I.

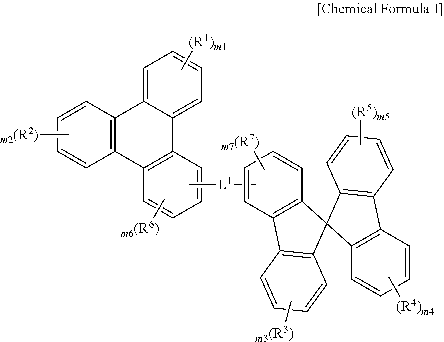

In Chemical Formula I, L¹ may be or may include, e.g., a single bond, a substituted or unsubstituted C6 to C20 arylene group, a substituted or unsubstituted C2 to C20 heterocyclic group, or a combination thereof.

R¹ to R⁷ may each independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof.

m1 to m5 may each independently be, e.g., an integer of 1 to 4.

m6 and m7 may each independently be, e.g., an integer of 1 to 3.

In an implementation, Chemical Formula I may satisfy or meet at least one of the following conditions (i) and (ii).

(i) L¹ may be, e.g., a C6 to C20 arylene group substituted with at least one deuterium or a C2 to C20 heterocyclic group substituted with at least one deuterium.

(ii) at least one of R¹ to R⁷ may be, e.g., deuterium, a C6 to C30 aryl group substituted with at least one deuterium, or a C2 to C30 heterocyclic group substituted with at least one deuterium.

The compound represented by Chemical Formula I has a structure in which triphenylene is substituted with spirofluorene as a basic skeleton, and has a structure in which the basic skeleton is substituted with at least one deuterium.

The zero-point energy and vibrational energy of the compound may be lower as at least one deuterium is substituted. As a result, the energy of the ground state may be further lowered, and the thin film formed therefrom may be made into an amorphous state due to the weakening of intermolecular interaction, which may help further improve heat resistance and is effective in improving life-span. In an implementation, when this is applied, a high-efficiency, particularly long life-span organic light emitting diode may be realized.

In an implementation, L¹ may be, e.g., a single bond and at least one of R¹ to R⁷ may be deuterium, a C6 to C30 aryl group substituted with at least one deuterium, or a C2 to C30 heterocyclic group substituted with at least one deuterium.

In an implementation, L¹ may be, e.g., a C6 to C20 arylene group substituted with at least one deuterium or a C2 to C20 heterocyclic group substituted with at least one deuterium and simultaneously at least one of R¹ to R⁷ may be deuterium, a C6 to C30 aryl group substituted with at least one deuterium, or a C2 to C30 heterocyclic group substituted with at least one deuterium.

In an implementation, L¹ may be, e.g., a phenylene group substituted with at least one deuterium, a biphenylene group substituted with at least one deuterium, a terphenylene group substituted with at least one deuterium, a naphthylene group substituted with at least one deuterium, an anthracenylene group substituted with at least one deuterium, a phenanthrenylene group substituted with at least one deuterium, a triphenylenylene group substituted with at least one deuterium, a fluorenylene group substituted with at least one deuterium, a carbazolylene group substituted with at least one deuterium, a dibenzofuranylene group substituted with at least one deuterium, or a dibenzothiophenylene group substituted with at least one deuterium.

In an implementation, L¹ may be, e.g., a single bond, a phenylene group substituted with at least one deuterium, a naphthylene group substituted with at least one deuterium, a carbazolylene group substituted with at least one deuterium, dibenzofuranylene group substituted with at least one deuterium, or a dibenzothiophenylene group substituted with at least one deuterium.

In an implementation, at least one of R¹ to R⁷ may be, e.g., deuterium, a phenyl group substituted with at least one deuterium, a biphenyl group substituted with at least one deuterium, a terphenyl group substituted with at least one deuterium, a naphthyl group substituted with at least one deuterium, an anthracenyl group substituted with at least one deuterium, a phenanthrenyl group substituted with at least one deuterium, a triphenylene group substituted with at least one deuterium, a fluorenyl group substituted with at least one deuterium, a carbazolyl group substituted with at least one deuterium, a dibenzofuranyl group substituted with at least one deuterium, or a dibenzothiophenyl group substituted with at least one deuterium.

In an implementation, at least one of R¹ to R⁷ may be, e.g., deuterium, a phenyl group substituted with at least one deuterium, a carbazolyl group substituted with at least one deuterium, a dibenzofuranyl group substituted with at least one deuterium, or a dibenzothiophenyl group substituted with at least one deuterium.

In an implementation, the compound represented by Chemical Formula I may be, e.g., represented by any one of Chemical Formula I-1 to Chemical Formula I-4 according to a specific substitution point of spirofluorene for triphenylene.

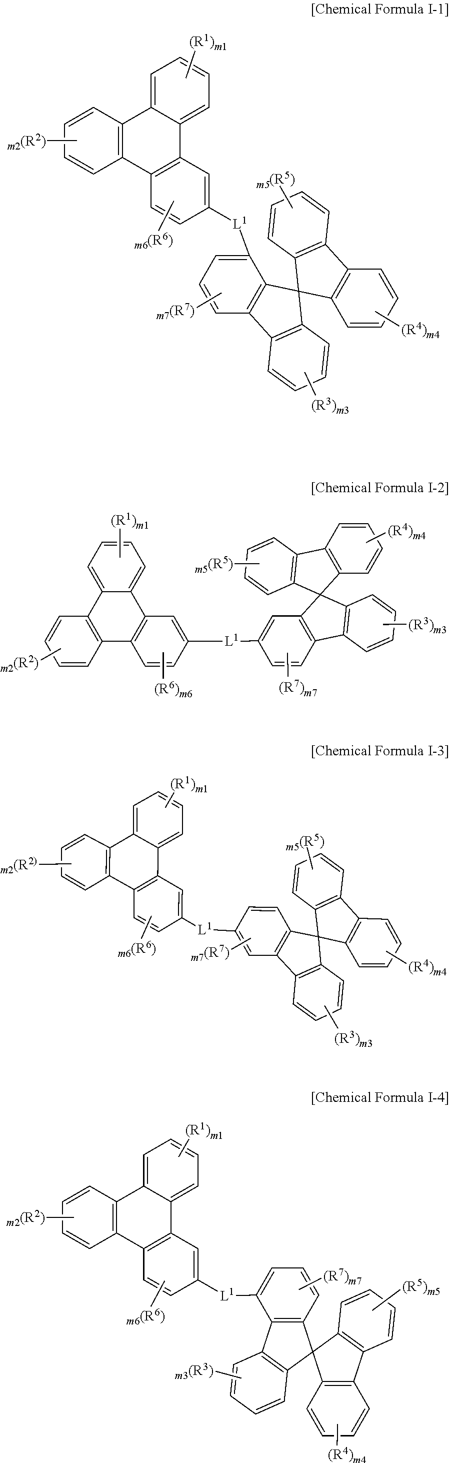

In Chemical Formula I-1 to Chemical Formula I-4, L¹, R¹ to R⁷, and m1 to m7 may be defined the same as those of Chemical Formula I described above.

In an implementation, the compound represented by Chemical Formula I may be represented by Chemical Formula I-4.

In an implementation, L¹ of Chemical Formula I-4 may be, e.g., a single bond or a phenylene group substituted with at least one deuterium.

In an implementation, R¹ to R⁷ in Chemical Formula I-4 may each independently be, e.g., hydrogen, deuterium, a substituted or unsubstituted phenyl group, or a substituted or unsubstituted carbazolyl group, and at least one of R¹ to R⁷ may be, e.g., deuterium, a phenyl group substituted with at least one deuterium, or a carbazolyl group substituted with at least one deuterium.

In an implementation, L¹ of Chemical Formula I-4 may be, e.g., a meta-phenylene group substituted with at least one deuterium or a para-phenylene group substituted with at least one deuterium.

In an implementation, L¹ may be, e.g., a phenylene group substituted with deuterium, e.g., may be one of the linking groups of Group I.

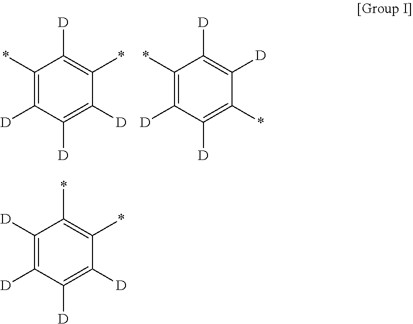

In Group I, * is a linking point.

In an implementation, at least two of R¹ to R⁷ may be deuterium.

In an implementation, four R¹'s may be deuterium.

In an implementation, three R¹'s may be deuterium, and the other one may be a phenyl group substituted with at least one deuterium, a biphenyl group substituted with at least one deuterium, or a carbazolyl group substituted with at least one deuterium.

In an implementation, four R²'s may be deuterium.

In an implementation, three R²'s may be deuterium, and the remaining one may be a phenyl group substituted with at least one deuterium, a biphenyl group substituted with at least one deuterium, or a carbazolyl group substituted with at least one deuterium.

In an implementation, four R³'s may be deuterium.

In an implementation, three R³'s may be deuterium, and the remaining one may be a phenyl group substituted with at least one deuterium, a biphenyl group substituted with at least one deuterium, or a carbazolyl group substituted with at least one deuterium.

In an implementation, four R⁴'s may be deuterium.

In an implementation, three R⁴'s may be deuterium, and the remaining one may be a phenyl group substituted with at least one deuterium, a biphenyl group substituted with at least one deuterium, or a carbazolyl group substituted with at least one deuterium.

In an implementation, four R⁵'s may be deuterium.

In an implementation, three R⁵'s may be deuterium, and the other one may be a phenyl group substituted with at least one deuterium, a biphenyl group substituted with at least one deuterium, or a carbazolyl group substituted with at least one deuterium.

In an implementation, three R⁶'s may be deuterium.

In an implementation, two R⁶'s may be deuterium, and the other one may be a phenyl group substituted with at least one deuterium, a biphenyl group substituted with at least one deuterium, or a carbazolyl group substituted with at least one deuterium.

In an implementation, three R⁷'s may be deuterium.

In an implementation, two R⁷'s may be deuterium, and the other one may be a phenyl group substituted with at least one deuterium, a biphenyl group substituted with at least one deuterium, or a carbazolyl group substituted with at least one deuterium.

In an implementation, R¹ to R⁷ may each be deuterium, m1 to m5 may each be 4, and m6 and m7 may each be 3.

In an implementation, L¹ may be, e.g., a phenylene group substituted with at least one deuterium, a naphthylene group substituted with at least one deuterium, or a carbazolylene group substituted with at least one deuterium, and at least one of R¹ to R⁷ may be, e.g., a phenyl group substituted with at least one deuterium or a carbazolyl group substituted with at least one deuterium and the remaining one may be deuterium or a phenyl group.

In an implementation, L¹ may be, e.g., a phenylene group all substituted with deuterium, a naphthylene group substituted with at least one deuterium, or a carbazolylene group all substituted with deuterium, and R¹ to R⁷ may each independently be, e.g., deuterium, a phenyl group all substituted with deuterium, or a carbazolyl group all substituted with deuterium.

In an implementation, the compound for an organic optoelectronic device represented by Chemical Formula I may be a compound of Group 1.

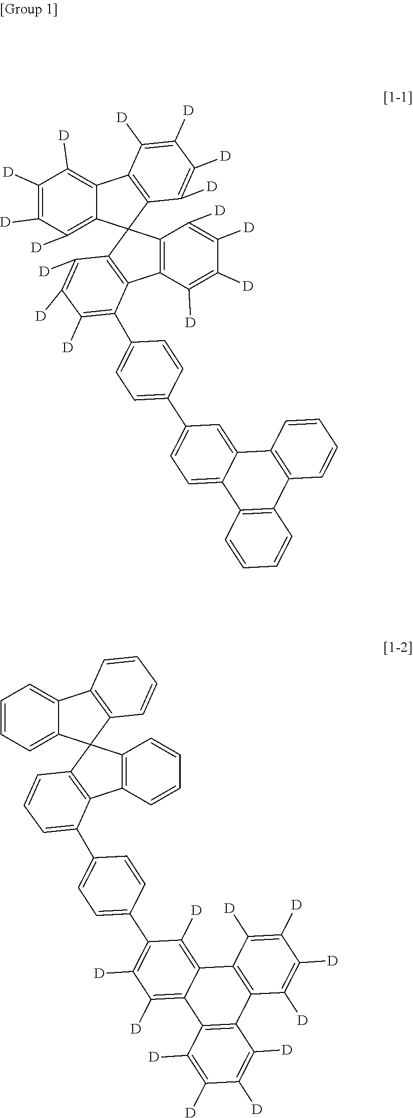 [SMILES: [2H]c1c([2H])c([2H])c2c(c1[2H])-c1c([2H])c([2H])c([2H])c([2H])c1C21c2c([2H])c([2H])c([2H])c([2H])c2-c2c(-c3ccc(-c4ccc5c6ccccc6c6ccccc6c5c4)cc3)c([2H])c([2H])c([2H])c21 ; [2H]c1c([2H])c([2H])c2c(c1[2H])c1c([2H])c([2H])c([2H])c([2H])c1c1c([2H])c(-c3ccc(-c4cccc5c4-c4ccccc4C54c5ccccc5-c5ccccc54)cc3)c([2H])c([2H])c21]

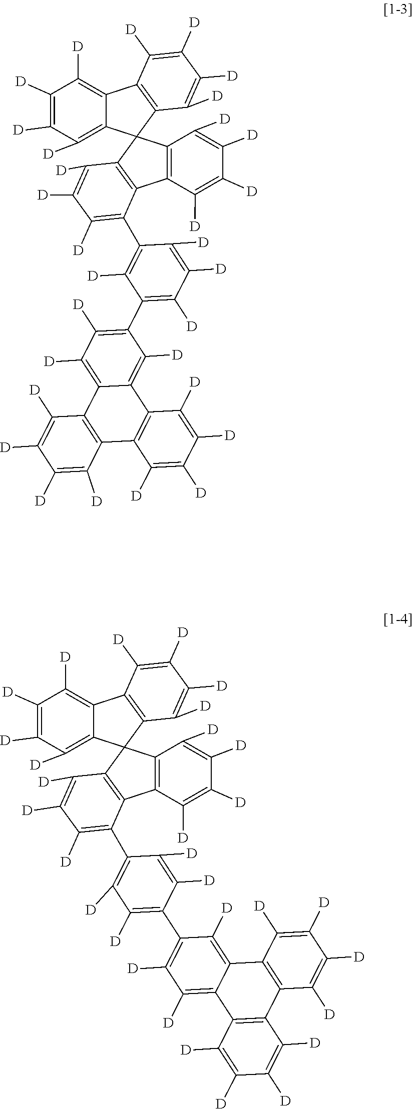

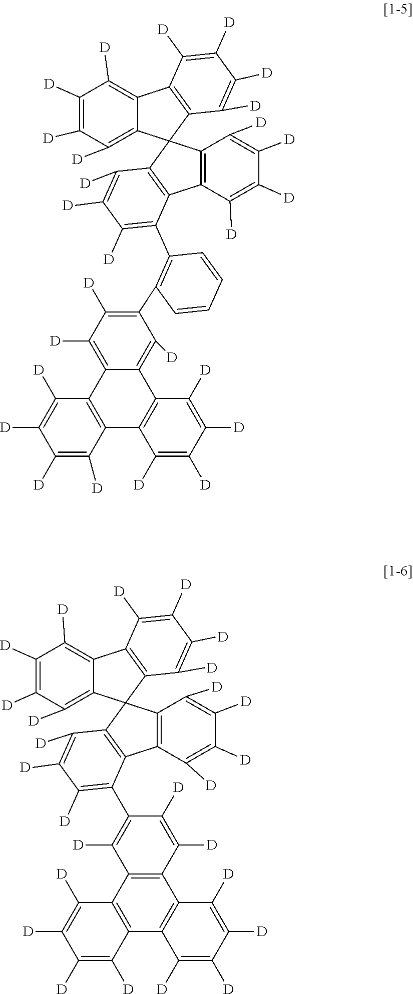 [SMILES: [2H]c1c([2H])c([2H])c2c(c1[2H])-c1c([2H])c([2H])c([2H])c([2H])c1C21c2c([2H])c([2H])c([2H])c([2H])c2-c2c(-c3ccccc3-c3c([2H])c([2H])c4c5c([2H])c([2H])c([2H])c([2H])c5c5c([2H])c([2H])c([2H])c([2H])c5c4c3[2H])c([2H])c([2H])c([2H])c21 ; [2H]c1c([2H])c([2H])c2c(c1[2H])-c1c([2H])c([2H])c([2H])c([2H])c1C21c2c([2H])c([2H])c([2H])c([2H])c2-c2c(-c3c([2H])c([2H])c4c5c([2H])c([2H])c([2H])c([2H])c5c5c([2H])c([2H])c([2H])c([2H])c5c4c3[2H])c([2H])c([2H])c([2H])c21]

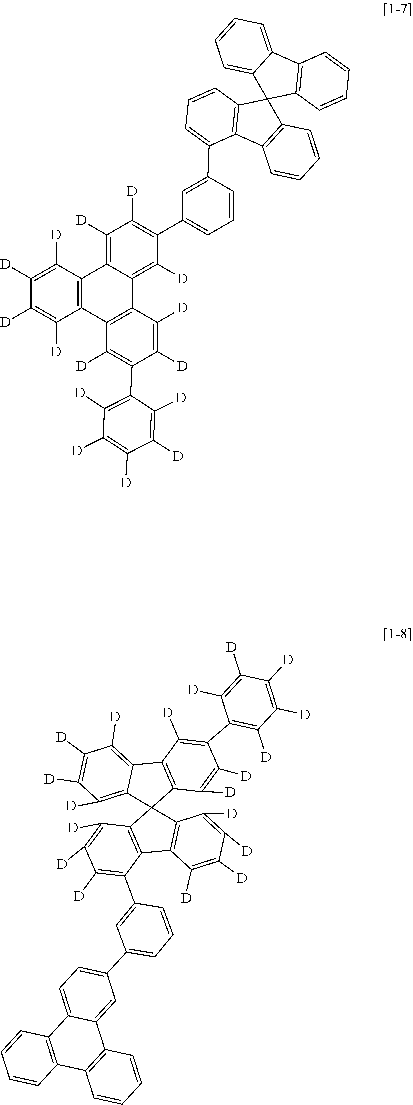 [SMILES: [2H]c1c([2H])c([2H])c(-c2c([2H])c([2H])c3c4c([2H])c(-c5cccc(-c6cccc7c6-c6ccccc6C76c7ccccc7-c7ccccc76)c5)c([2H])c([2H])c4c4c([2H])c([2H])c([2H])c([2H])c4c3c2[2H])c([2H])c1[2H] ; [2H]c1c([2H])c([2H])c(-c2c([2H])c([2H])c3c(c2[2H])-c2c([2H])c([2H])c([2H])c([2H])c2C32c3c([2H])c([2H])c([2H])c([2H])c3-c3c(-c4cccc(-c5ccc6c7ccccc7c7ccccc7c6c5)c4)c([2H])c([2H])c([2H])c32)c([2H])c1[2H]]

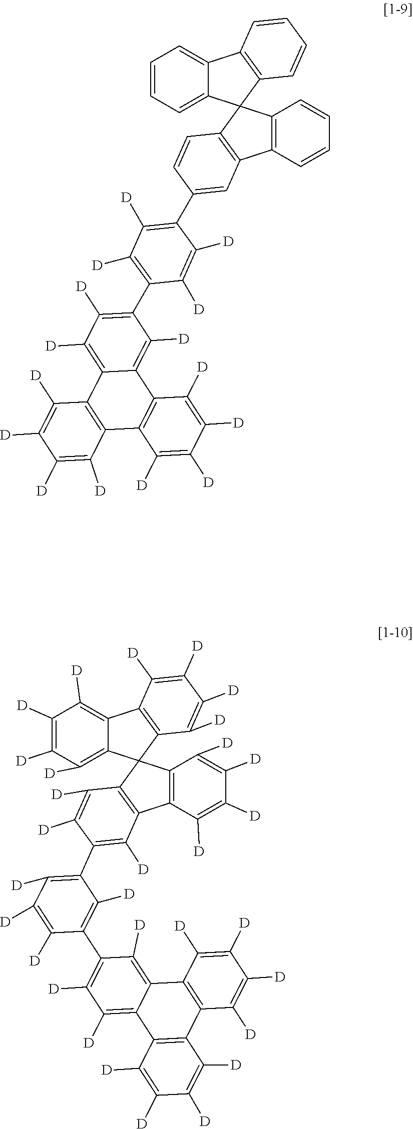 [SMILES: [2H]c1c([2H])c(-c2c([2H])c([2H])c3c4c([2H])c([2H])c([2H])c([2H])c4c4c([2H])c([2H])c([2H])c([2H])c4c3c2[2H])c([2H])c([2H])c1-c1ccc2c(c1)-c1ccccc1C21c2ccccc2-c2ccccc21 ; [2H]c1c([2H])c(-c2c([2H])c([2H])c3c(c2[2H])-c2c([2H])c([2H])c([2H])c([2H])c2C32c3c([2H])c([2H])c([2H])c([2H])c3-c3c([2H])c([2H])c([2H])c([2H])c32)c([2H])c(-c2c([2H])c([2H])c3c4c([2H])c([2H])c([2H])c([2H])c4c4c([2H])c([2H])c([2H])c([2H])c4c3c2[2H])c1[2H]]

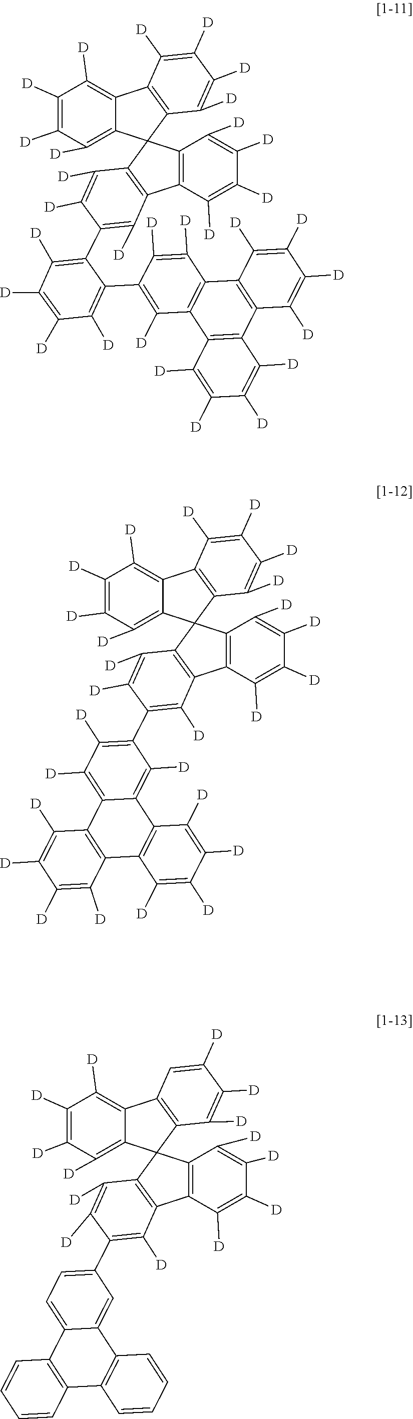

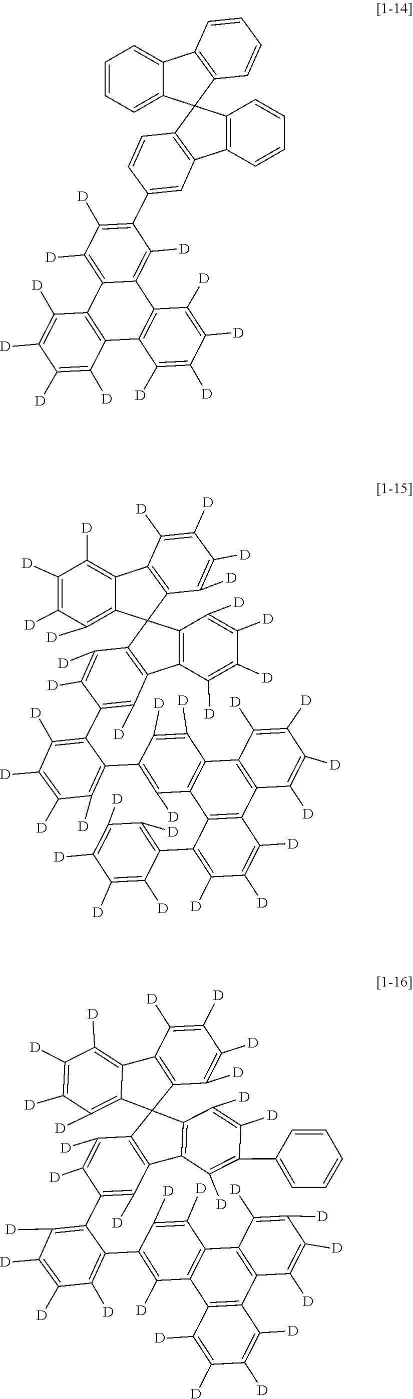

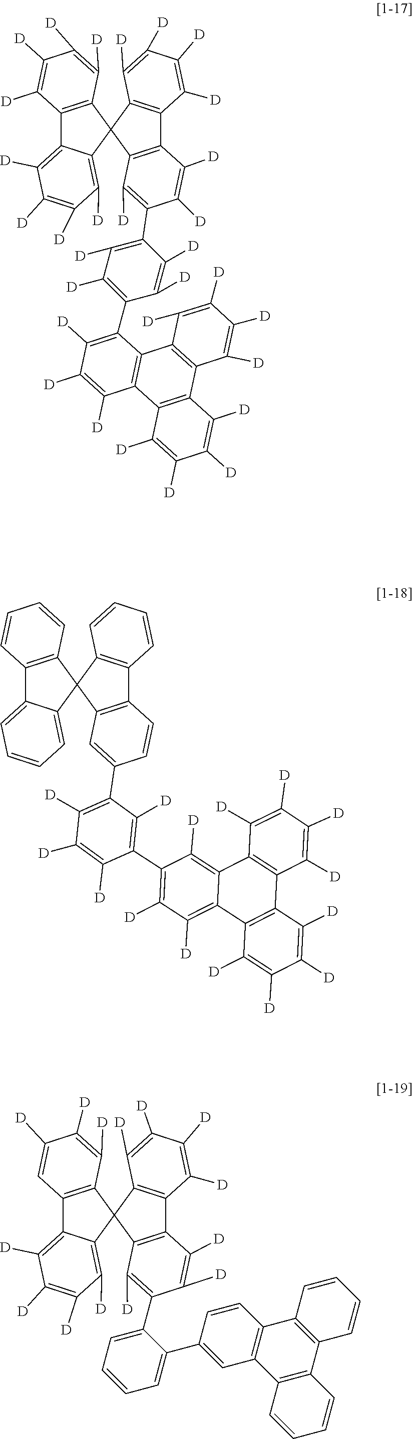

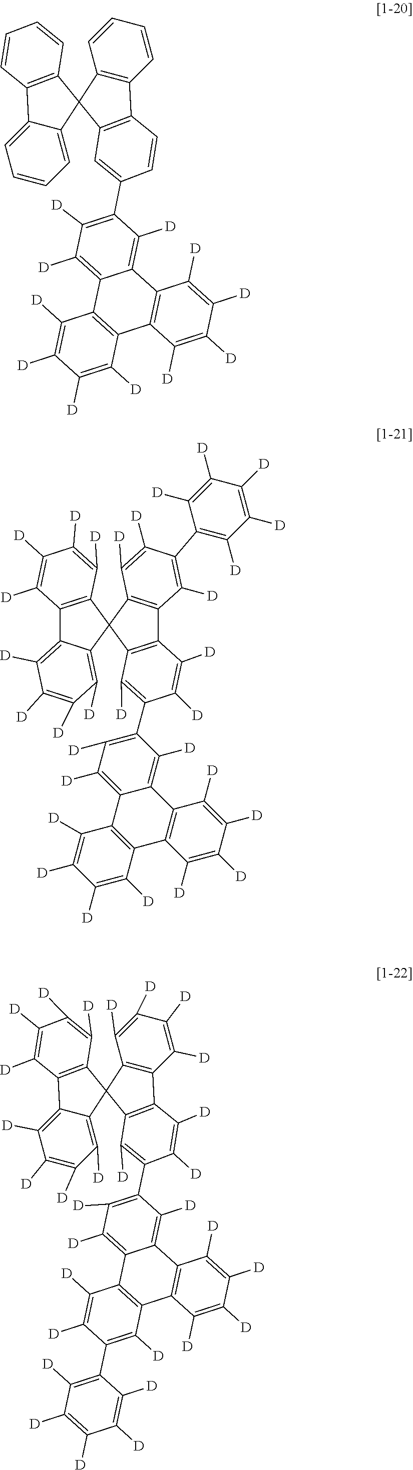

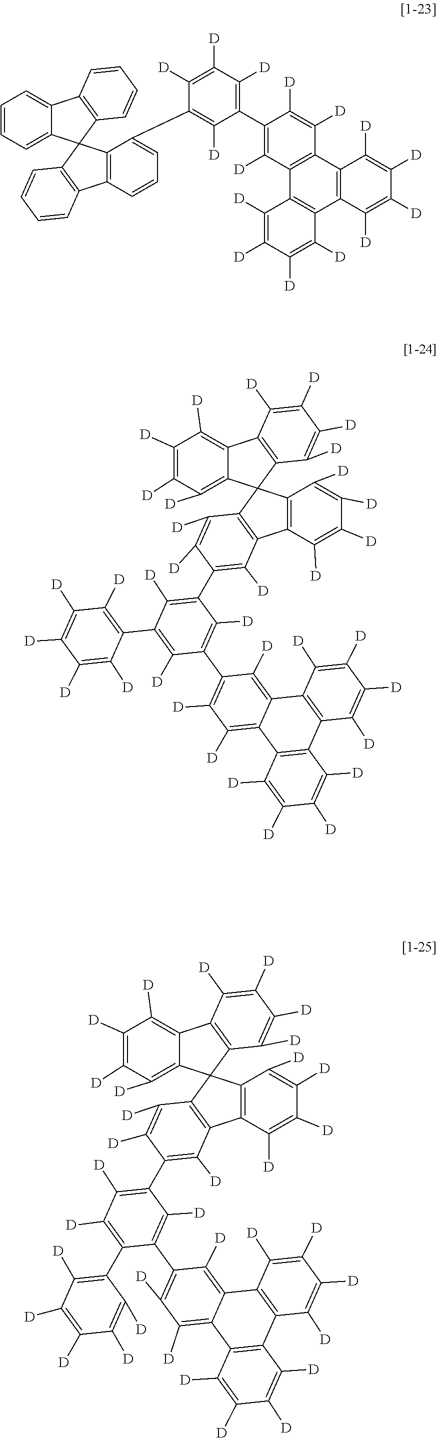

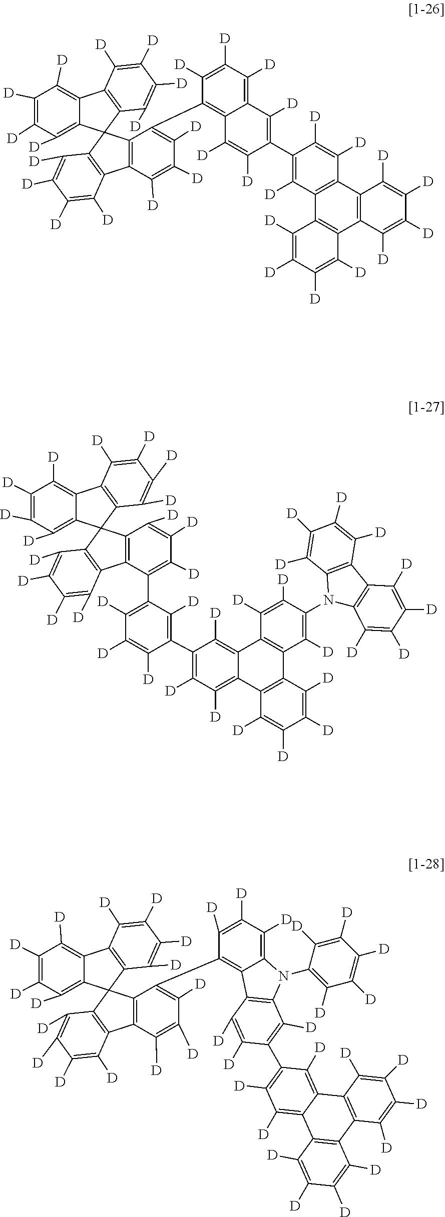

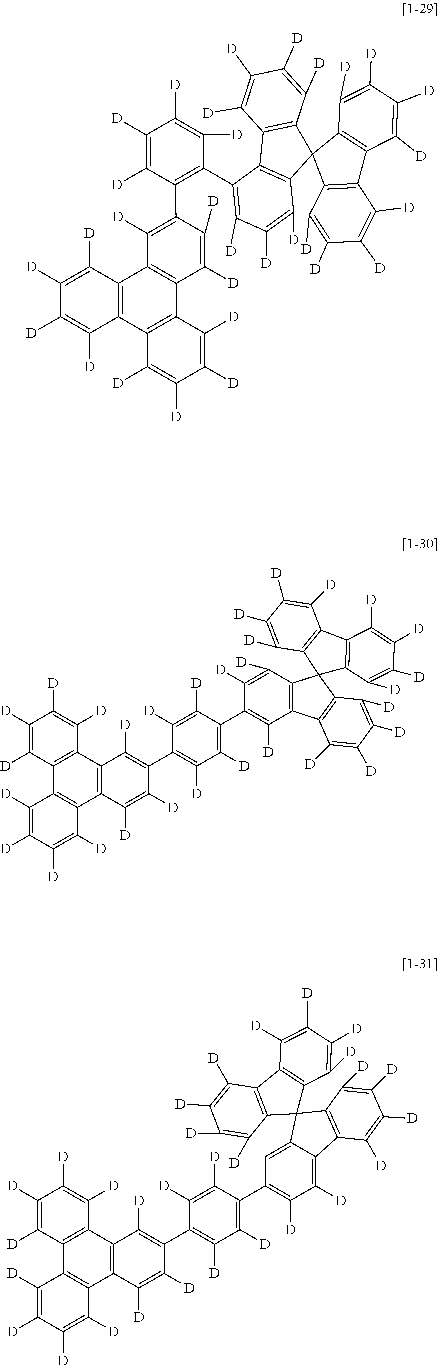

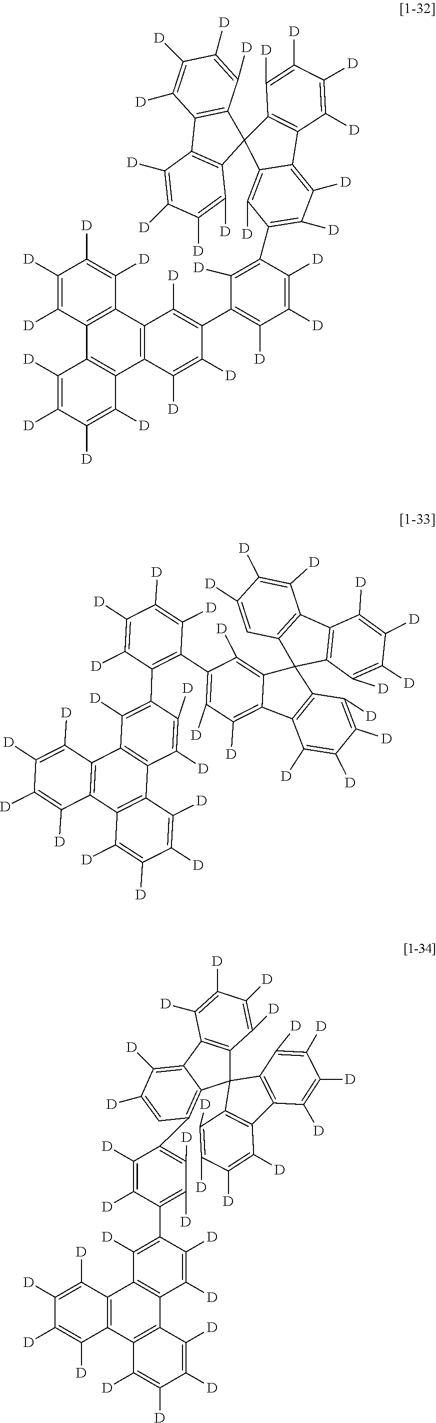

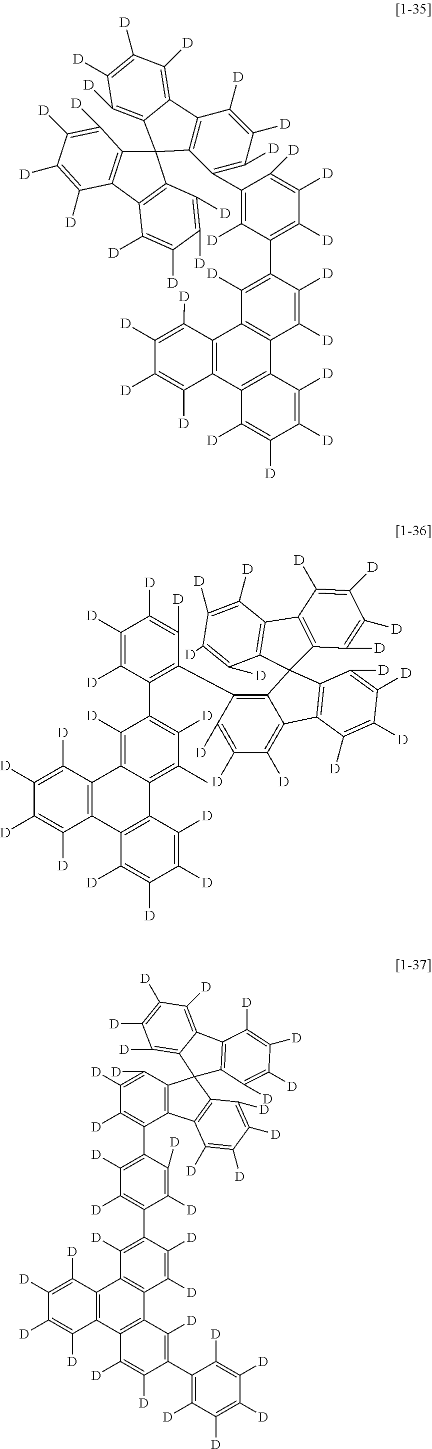

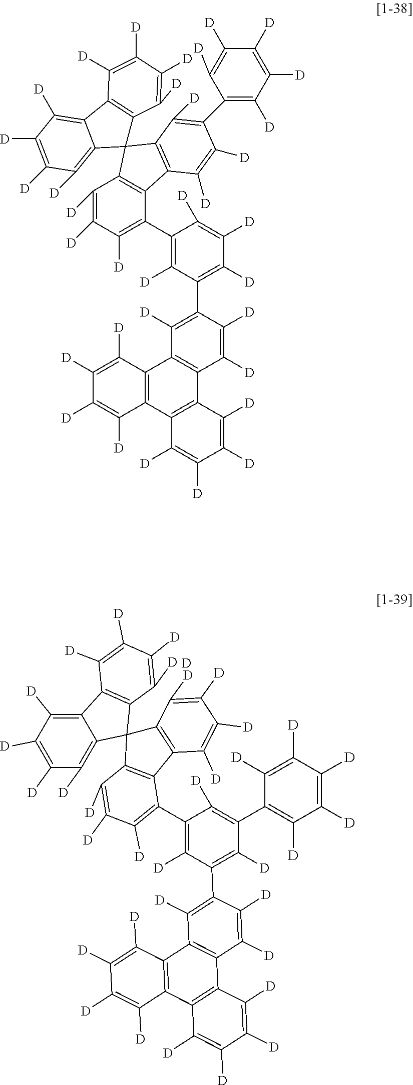

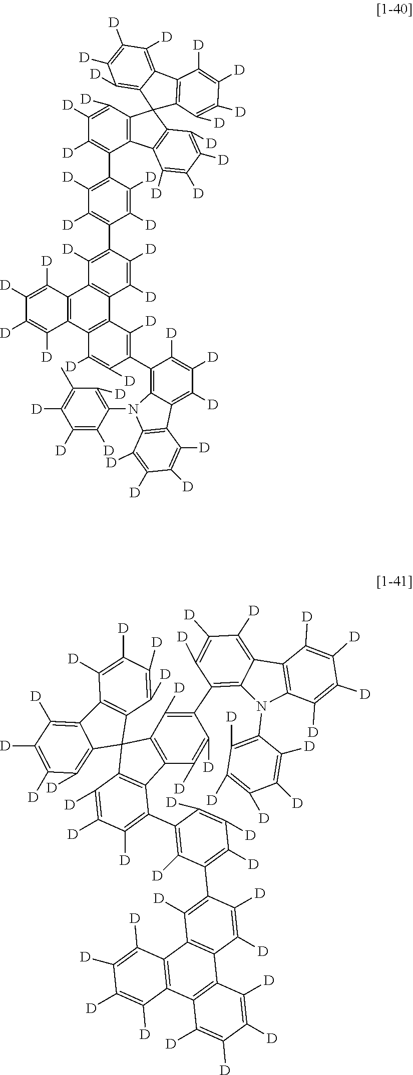

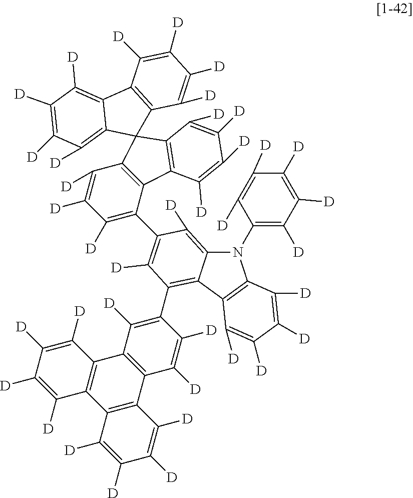 [SMILES: [2H]c1c([2H])c([2H])c(-n2c3c([2H])c([2H])c([2H])c([2H])c3c3c(-c4c([2H])c([2H])c5c6c([2H])c([2H])c([2H])c([2H])c6c6c([2H])c([2H])c([2H])c([2H])c6c5c4[2H])c([2H])c(-c4c([2H])c([2H])c([2H])c5c4-c4c([2H])c([2H])c([2H])c([2H])c4C54c5c([2H])c([2H])c([2H])c([2H])c5-c5c([2H])c([2H])c([2H])c([2H])c54)c([2H])c32)c([2H])c1[2H]]

In addition to the aforementioned compound for the organic optoelectronic device, one or more compounds may be further included.

In an implementation, the aforementioned compound for the organic optoelectronic device may be applied in the form of a composition further including a suitable host material.

In an implementation, the aforementioned compound for the organic optoelectronic device may further include a dopant.

The dopant may be a phosphorescent dopant, e.g., a red or green phosphorescent dopant.

The dopant may be a material mixed with the compound for the organic optoelectronic device in a small amount to cause light emission and may be generally a material such as a metal complex that emits light by multiple excitation into a triplet or more. The dopant may be, e.g., an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

L²MX [Chemical Formula Z]

In Chemical Formula Z, M may be, e.g., a metal, and L² and X are the same as or different from each other, and may be, e.g., ligands forming a complex compound with M.

The M may be, e.g., Ir, Pt, Os, Ti, Zr, Hf, Eu, Tb, Tm, Fe, Co, Ni, Ru, Rh, Pd, or a combination thereof, and L² and X may be, e.g., a bidentate ligand.

Examples of the ligands represented by L² and X may be a ligand of Group A.

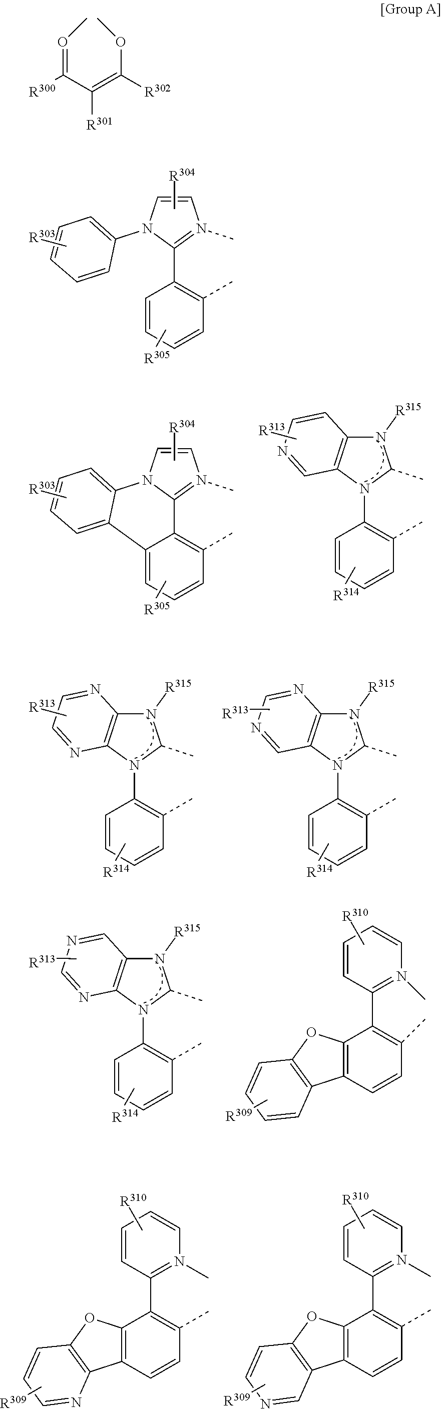

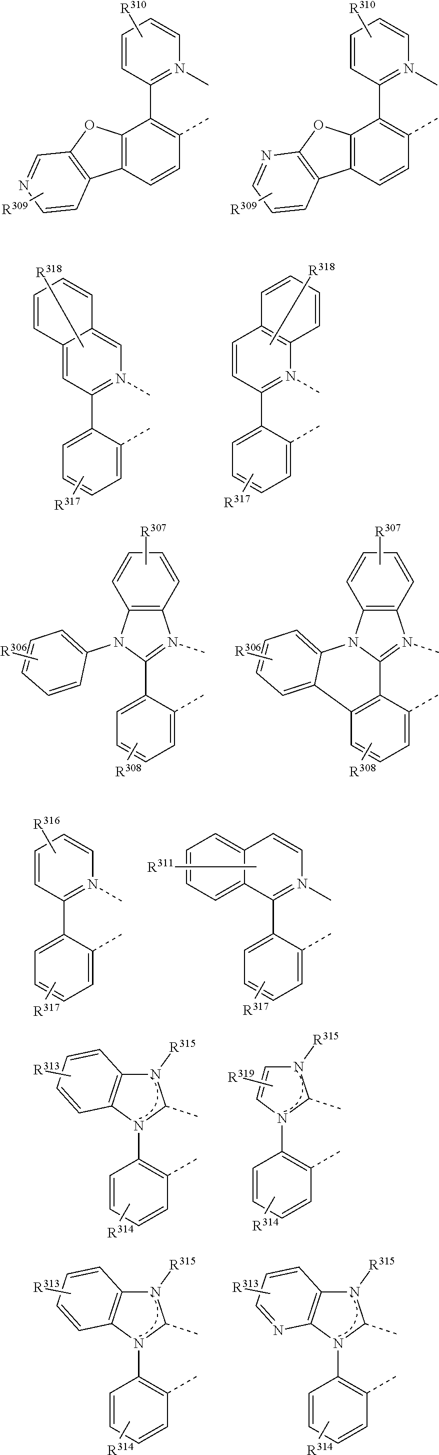

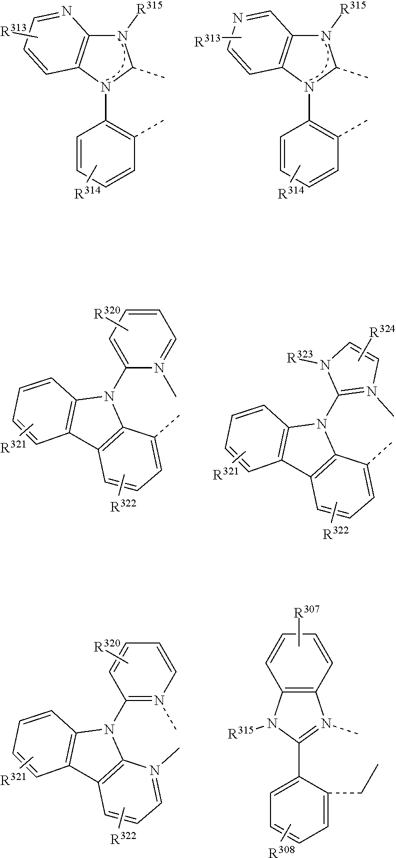

In Group A, R³⁰⁰ to R³⁰² may each independently be, e.g., hydrogen, deuterium, a C1 to C30 alkyl group that is substituted or unsubstituted with a halogen, a C6 to C30 aryl group that is substituted or unsubstituted with a C1 to C30 alkyl, or a halogen.

R³⁰³ to R³²⁴ may each independently be, e.g., hydrogen, deuterium, halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

In an implementation, it may include a dopant represented by Chemical Formula II.

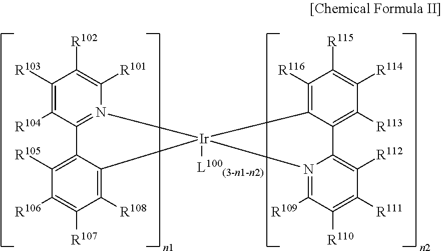

In Chemical Formula II, R¹⁰¹ to R¹¹⁶ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a C1 to C6 alkyl group.

In an implementation, at least one of R¹⁰¹ to R¹¹⁶ may be, e.g., a functional group represented by Chemical Formula II-1.

L¹⁰⁰ may be, e.g., a bidentate ligand of a monovalent anion, and is a ligand that coordinates to iridium through a lone pair of electrons of carbon or heteroatom,

n1 and n2 may each independently be, e.g., an integer 0 to 3, and n1+n2 may be an integer of 1 to 3,

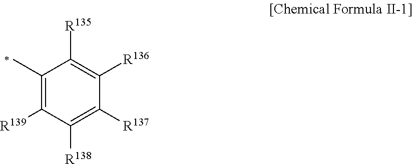

In Chemical Formula II-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

* means a portion linked to a carbon atom.

In an implementation, a dopant represented by Chemical Formula Z-1 may be included.

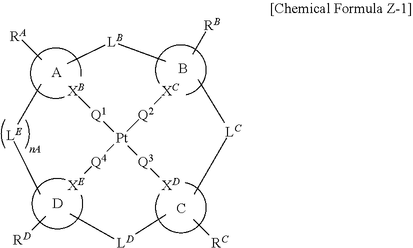

In Chemical Formula Z-1, rings A, B, C, and D may each independently represent a 5-membered or 6-membered carbocyclic or heterocyclic ring;

R^{A}, R^{B}, R^{C}, and R^{D} may each independently represent mono-, di-, tri-, or tetra-substitution, or unsubstitution;

L^{B}, L^{C}, and L^{D} may each independently be, e.g., a direct bond, BR, NR, PR, O, S, Se, C═O, S═O, SO₂, CRR′, SiRR′, GeRR′, and a combination thereof;

when nA is 1, L^{E} may be a direct bond, BR, NR, PR, O, S, Se, C═O, S═O, SO₂, CRR′, SiRR′, GeRR′, and a combination thereof; when nA is 0, L^{E} does not exist; and

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, and a combination thereof; any adjacent R^{A}, R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

In an implementation, the dopant according to an embodiment may be a platinum complex, and may be, e.g., represented by Chemical Formula III.

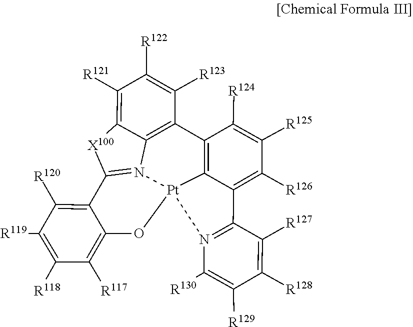

In Chemical Formula III, X¹⁰⁰ may be, e.g., O, S, or NR¹³¹.

R¹¹⁷ to R¹³¹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴,

R¹³² to R¹³⁴ may each independently be, e.g., a C1 to C6 alkyl group, and

at least one of R¹¹⁷ to R¹³¹ may be, e.g., —SiR¹³²R¹³³R¹³⁴ or a tert-butyl group.

Hereinafter, an organic optoelectronic device including the aforementioned compound for the organic optoelectronic device is described.

The organic optoelectronic device may be any device to convert electrical energy into photoenergy and vice versa without particular limitation, and may be, e.g., an organic photoelectric device, an organic light emitting diode, an organic solar cell, or an organic photo-conductor drum.

Herein, an organic light emitting diode as one example of an organic optoelectronic device is described referring to drawings.

The FIGURE is a cross-sectional view illustrating an organic light emitting diode according to an embodiment.

Referring to the FIGURE, an organic light emitting diode 100 according to an embodiment may include an anode 120 and a cathode 110 facing each other and an organic layer 105 between the anode 120 and cathode 110.

The anode 120 may be made of a conductor having a large work function to help hole injection, and may be, e.g., a metal, a metal oxide, or a conductive polymer. The anode 120 may be, e.g., a metal such as nickel, platinum, vanadium, chromium, copper, zinc, gold, or the like or an alloy thereof; a metal oxide such as zinc oxide, indium oxide, indium tin oxide (ITO), indium zinc oxide (IZO), or the like; a combination of a metal and an oxide such as ZnO and Al or SnO₂ and Sb; or a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof; or a multi-layer structure material such as LiF/Al, LiO₂/Al, LiF/Ca, or BaF₂/Ca.

The organic layer 105 may include the aforementioned compound for an organic optoelectronic device.

The organic layer 105 may include a light emitting layer 130 and the light emitting layer 130 may include the aforementioned compound for an organic optoelectronic device.

The composition for an organic optoelectronic device further including the dopant may be, e.g., a red or green light emitting composition.

The light emitting layer 130 may include, e.g., the aforementioned compound for an organic optoelectronic device.

The organic layer may further include a charge transport region in addition to the light emitting layer.

The charge transport region may be, e.g., a hole transport region 140.

The hole transport region 140 may help further increase hole injection and/or hole mobility between the anode 120 and the light emitting layer 130 and block electrons.

In an implementation, the hole transport region 140 may include a hole transport layer between the anode 120 and the light emitting layer 130, and a hole transport auxiliary layer between the light emitting layer 130 and the hole transport layer and a compound of Group B may be included in at least one of the hole transport layer and the hole transport auxiliary layer.

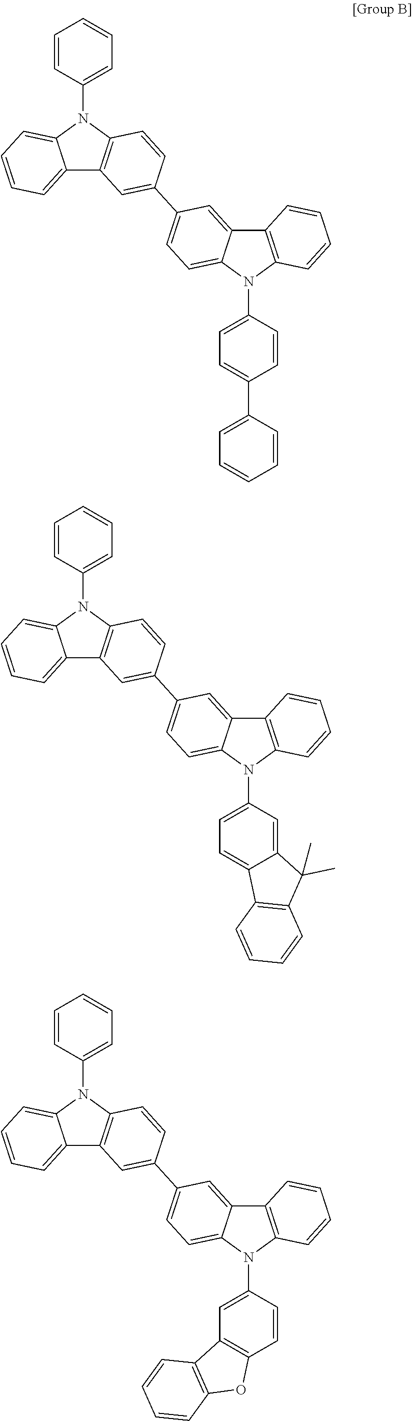 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

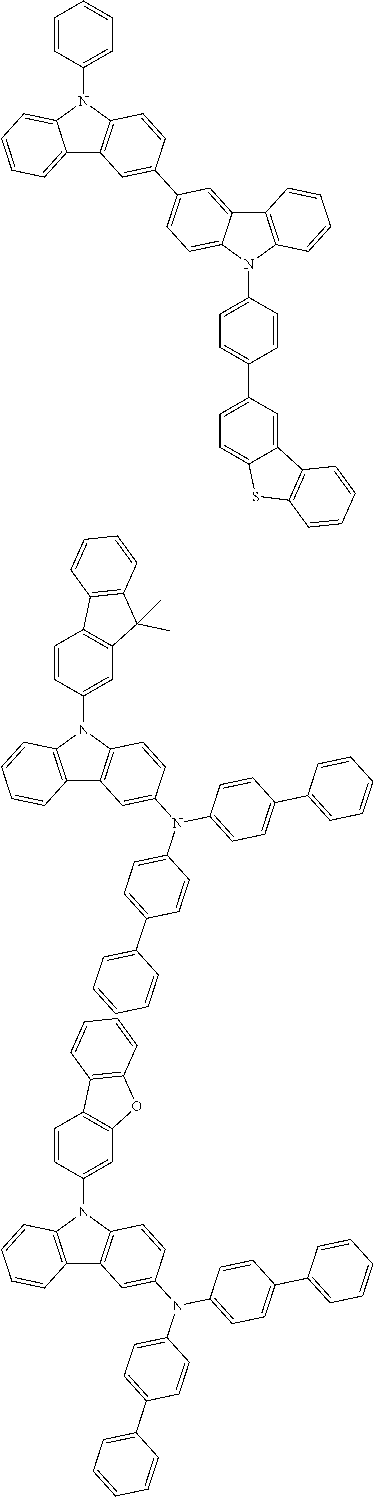

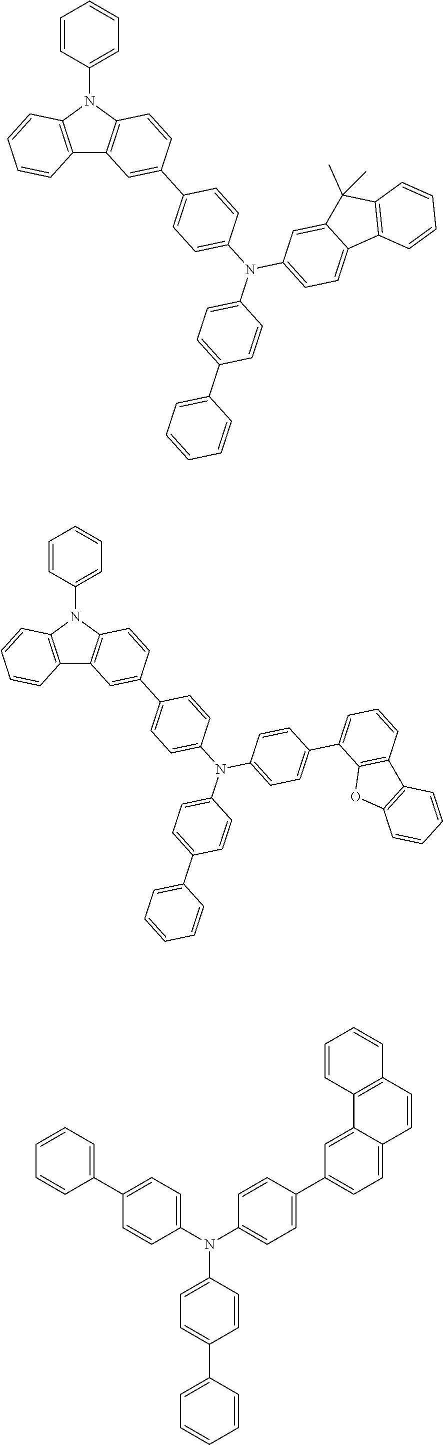 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5ccc6ccccc6c5c4)cc3)cc2)cc1]

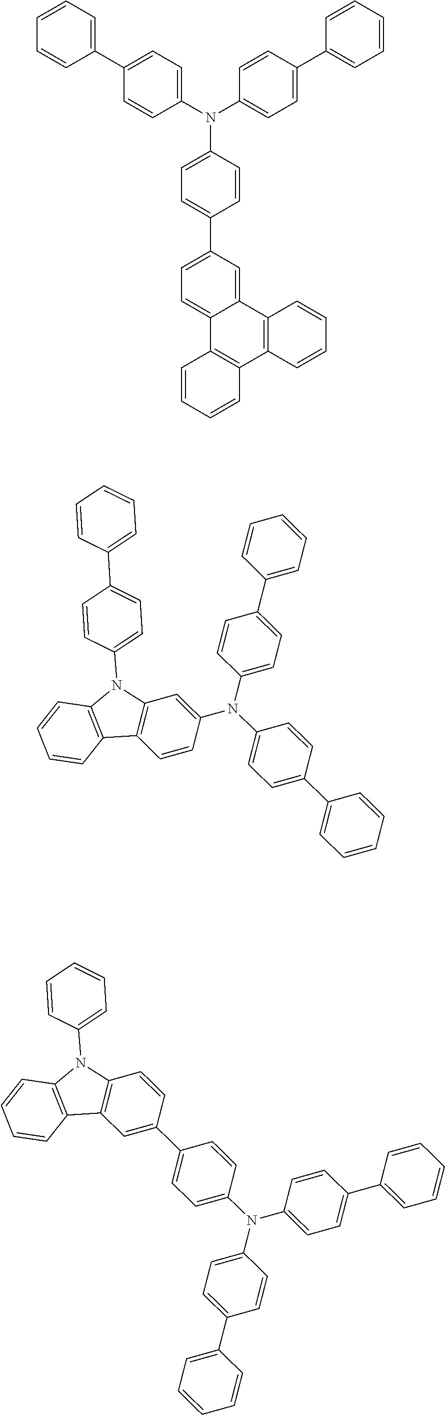 [SMILES: c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

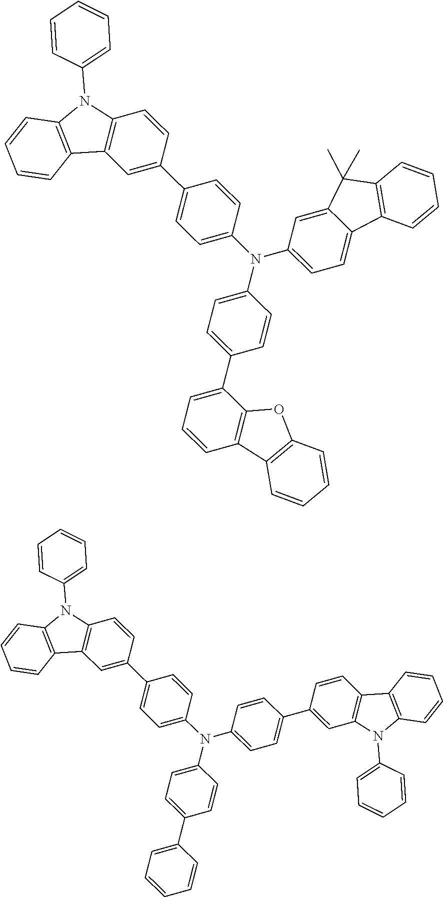 [SMILES: CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

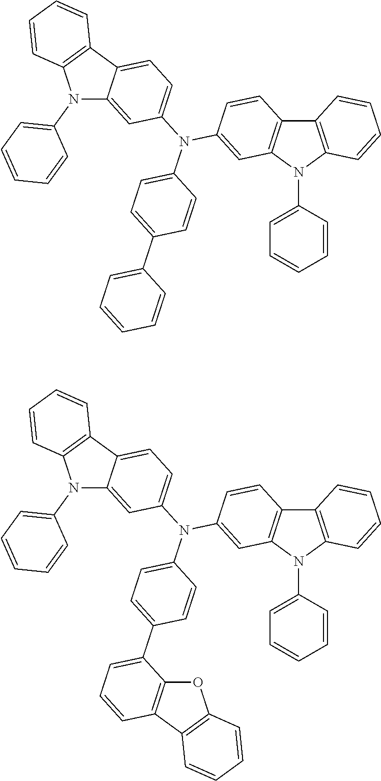 [SMILES: c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1]

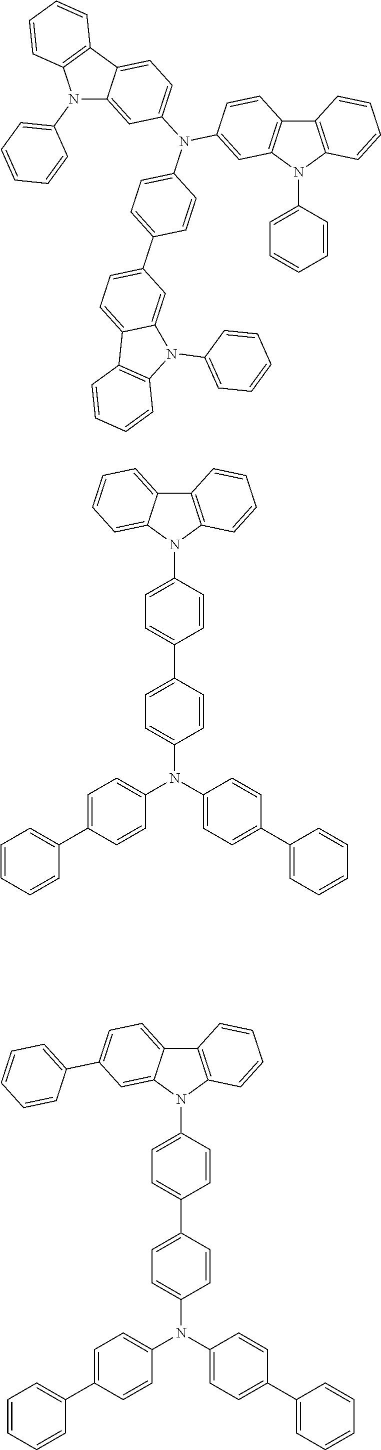

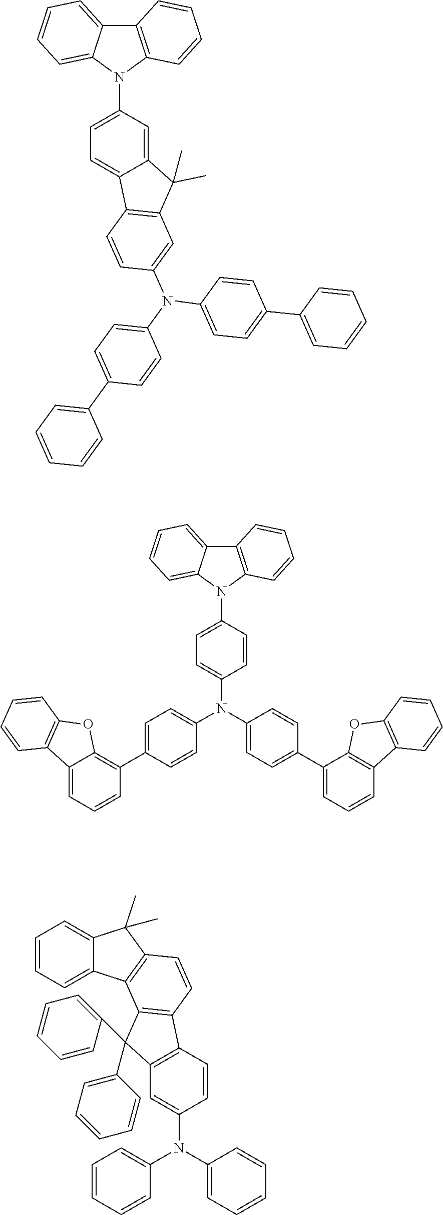 [SMILES: CC1(C)c2cc(N(c3ccc(-c4ccccc4)cc3)c3ccc(-c4ccccc4)cc3)ccc2-c2ccc(-n3c4ccccc4c4ccccc43)cc21 ; c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1]

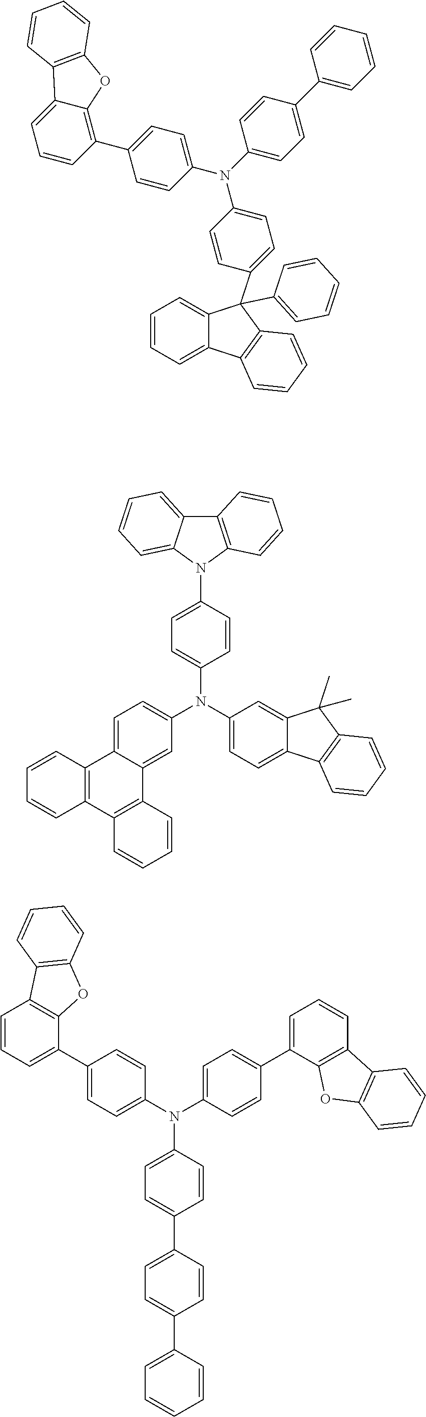 [SMILES: c1ccc(-c2ccc(N(c3ccc(-c4cccc5c4oc4ccccc45)cc3)c3ccc(C4(c5ccccc5)c5ccccc5-c5ccccc54)cc3)cc2)cc1 ; CC1(C)c2ccccc2-c2ccc(N(c3ccc(-n4c5ccccc5c5ccccc54)cc3)c3ccc4c5ccccc5c5ccccc5c4c3)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1]

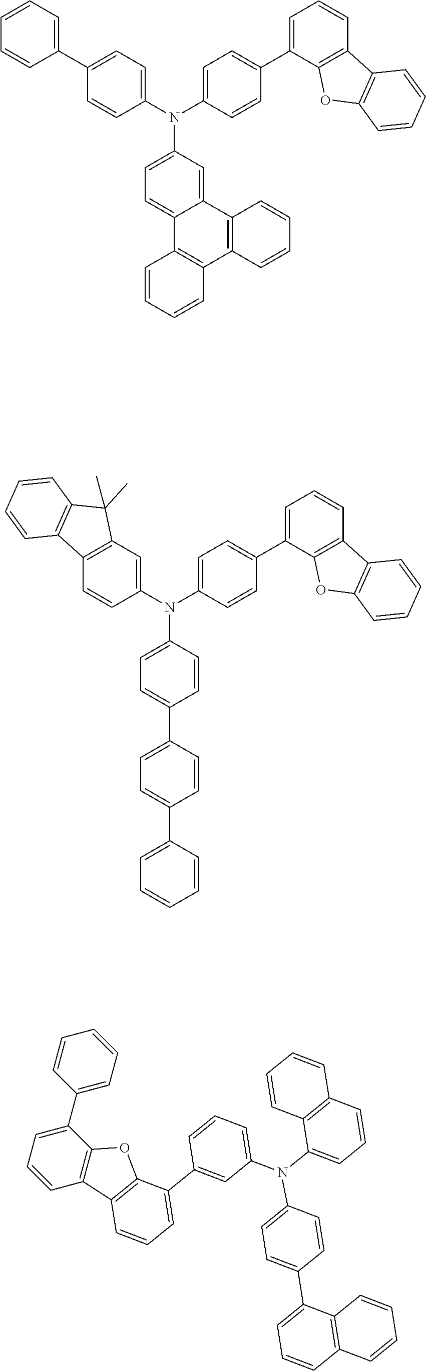 [SMILES: c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1]

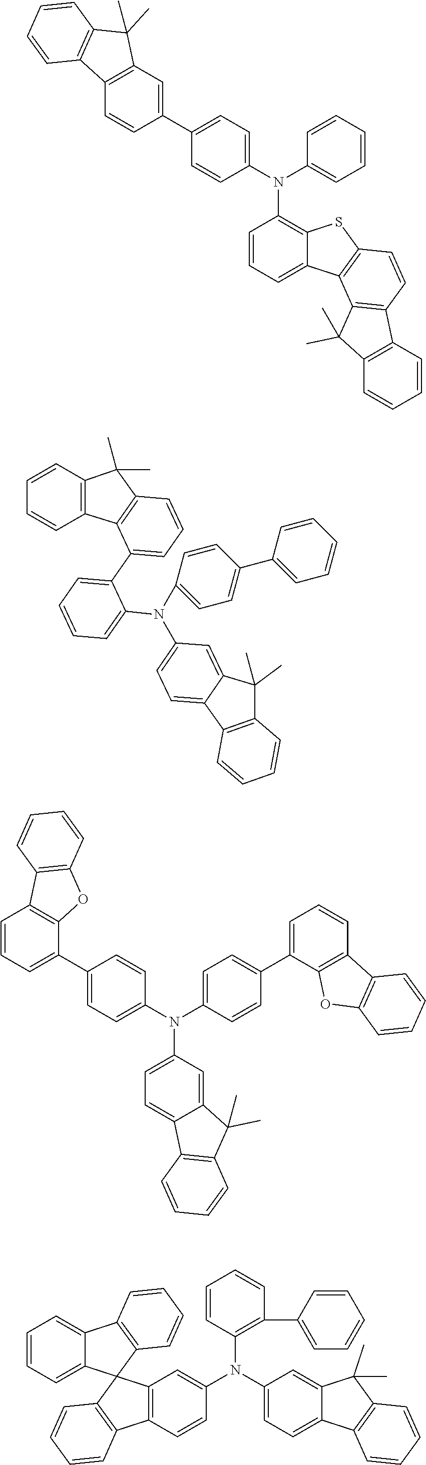 [SMILES: CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21 ; CC1(C)c2ccccc2-c2ccc(N(c3ccc(-c4cccc5c4oc4ccccc45)cc3)c3ccc(-c4cccc5c4oc4ccccc45)cc3)cc21 ; CC1(C)c2ccccc2-c2ccc(N(c3ccc4c(c3)C3(c5ccccc5-c5ccccc53)c3ccccc3-4)c3ccccc3-c3ccccc3)cc21]

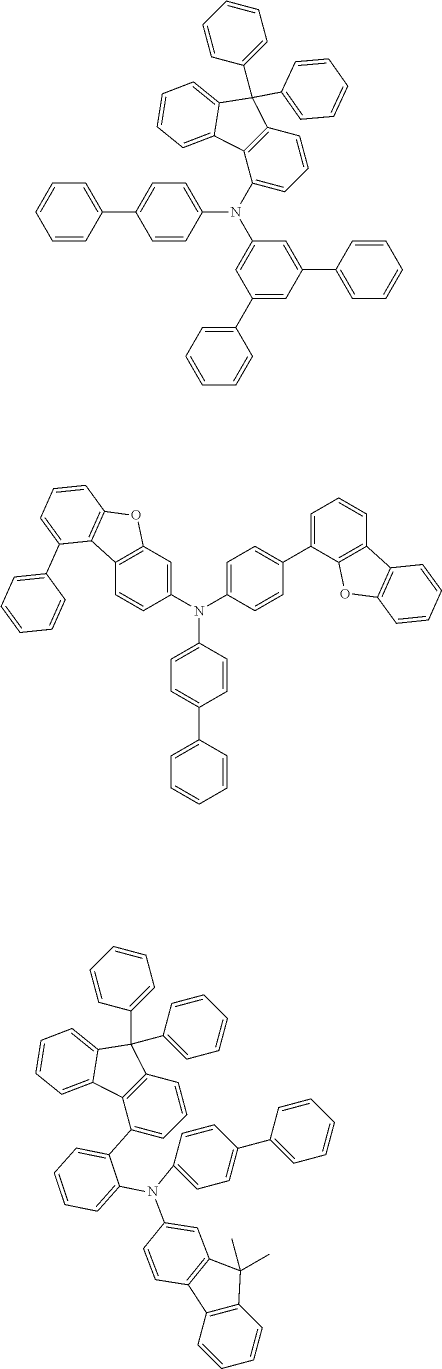 [SMILES: c1ccc(-c2ccc(N(c3cc(-c4ccccc4)cc(-c4ccccc4)c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21]

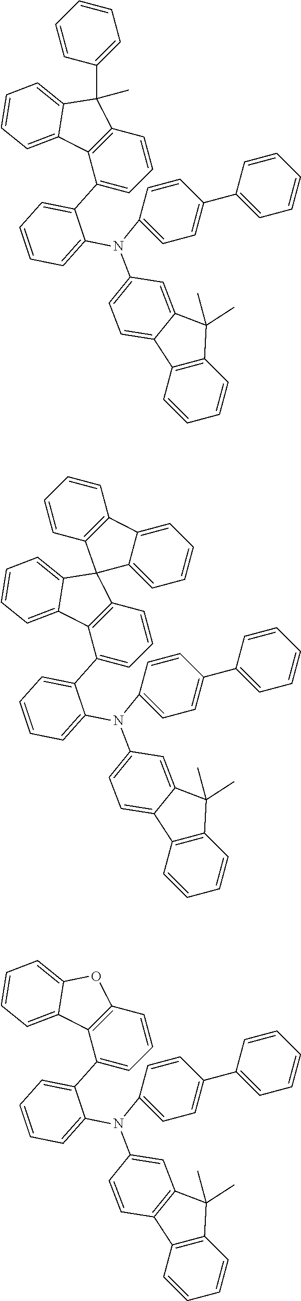 [SMILES: CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21]

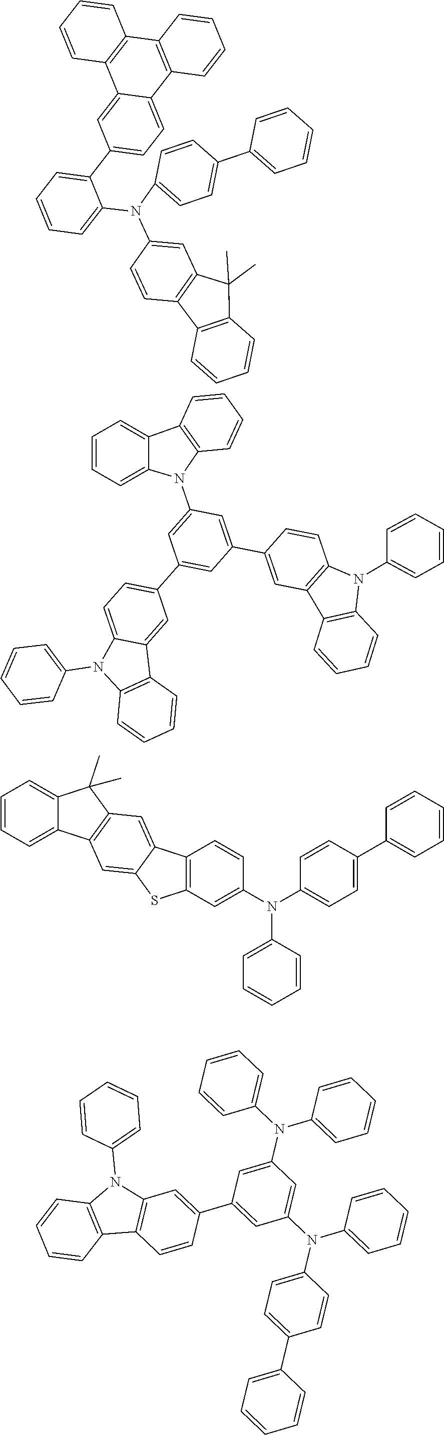

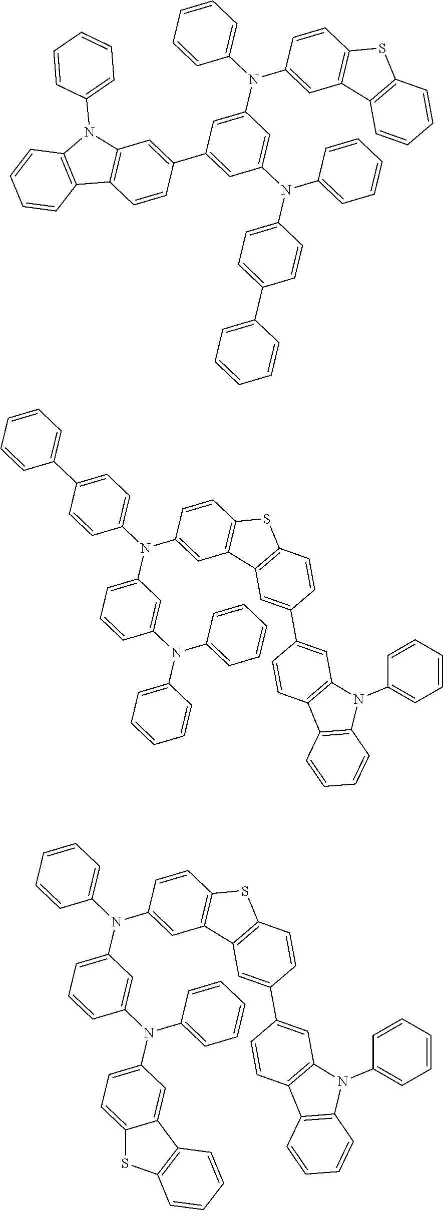 [SMILES: c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1]

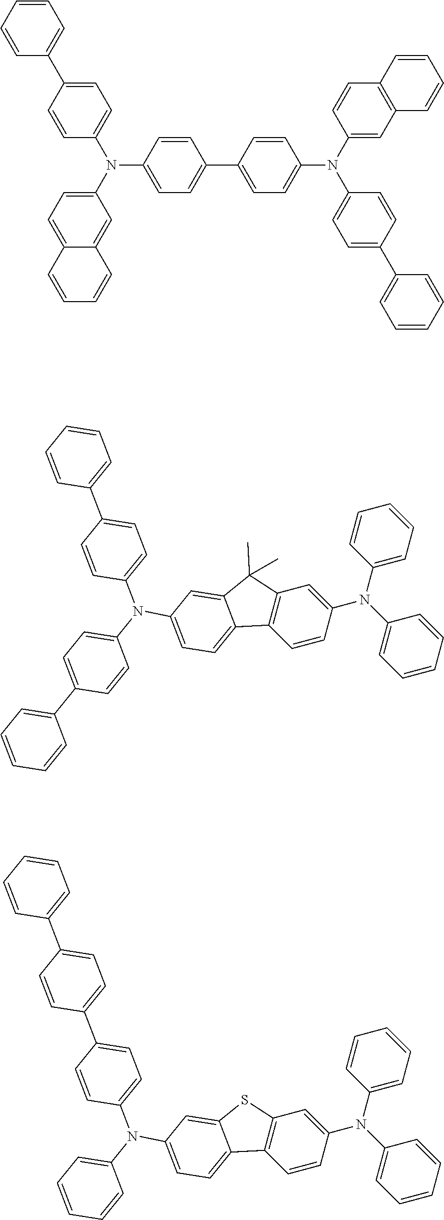 [SMILES: c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1 ; CC1(C)c2cc(N(c3ccccc3)c3ccccc3)ccc2-c2ccc(N(c3ccc(-c4ccccc4)cc3)c3ccc(-c4ccccc4)cc3)cc21 ; c1ccc(-c2ccc(-c3ccc(N(c4ccccc4)c4ccc5c(c4)sc4cc(N(c6ccccc6)c6ccccc6)ccc45)cc3)cc2)cc1]

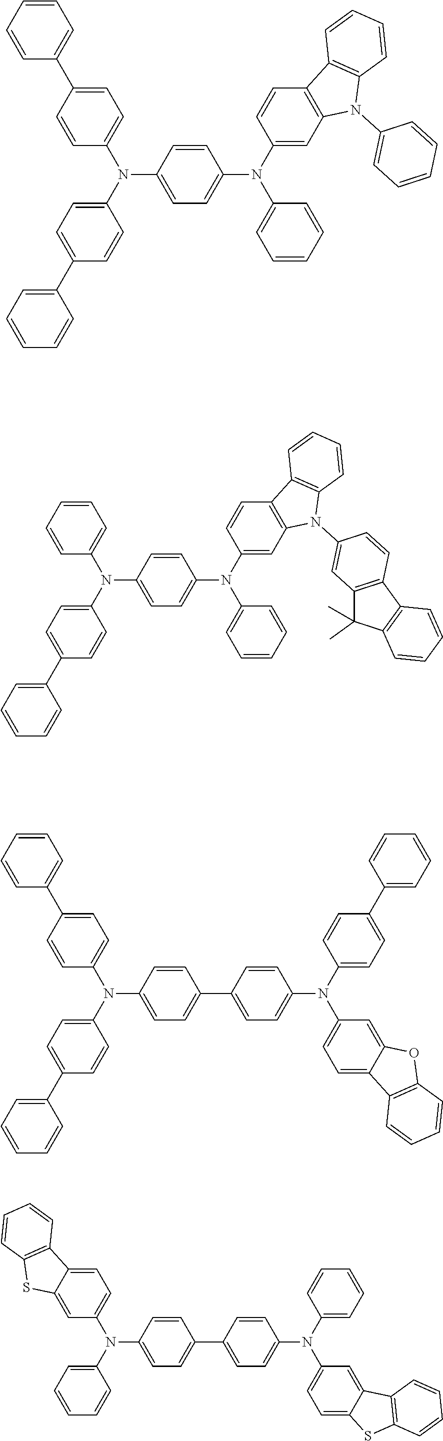 [SMILES: c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1 ; CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21 ; c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1]

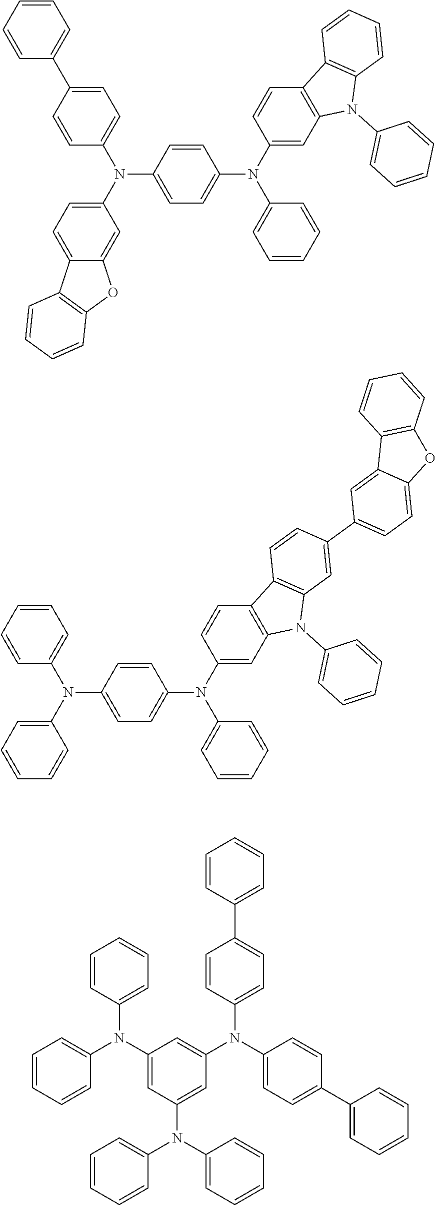 [SMILES: c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1 ; c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1 ; c1ccc(-c2ccc(N(c3ccc(-c4ccccc4)cc3)c3cc(N(c4ccccc4)c4ccccc4)cc(N(c4ccccc4)c4ccccc4)c3)cc2)cc1]

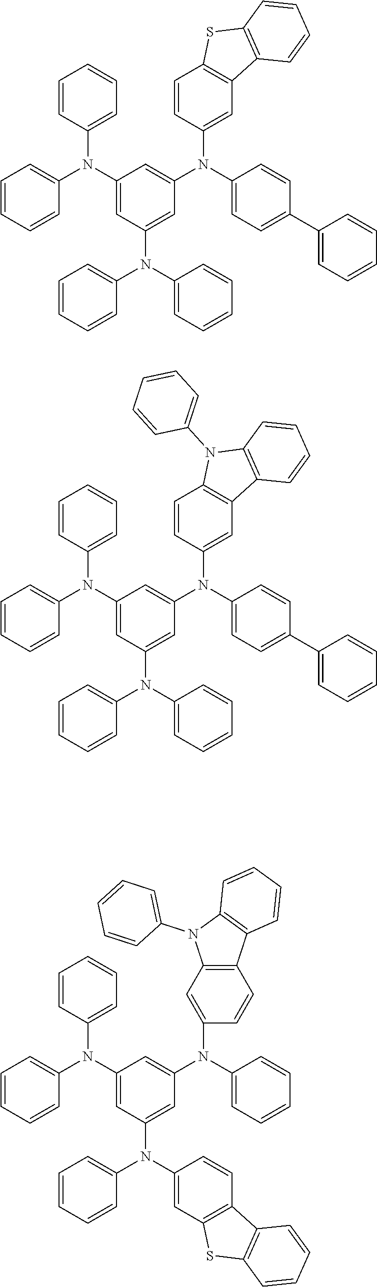 [SMILES: c1ccc(-c2ccc(N(c3cc(N(c4ccccc4)c4ccccc4)cc(N(c4ccccc4)c4ccccc4)c3)c3ccc4sc5ccccc5c4c3)cc2)cc1 ; c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1]

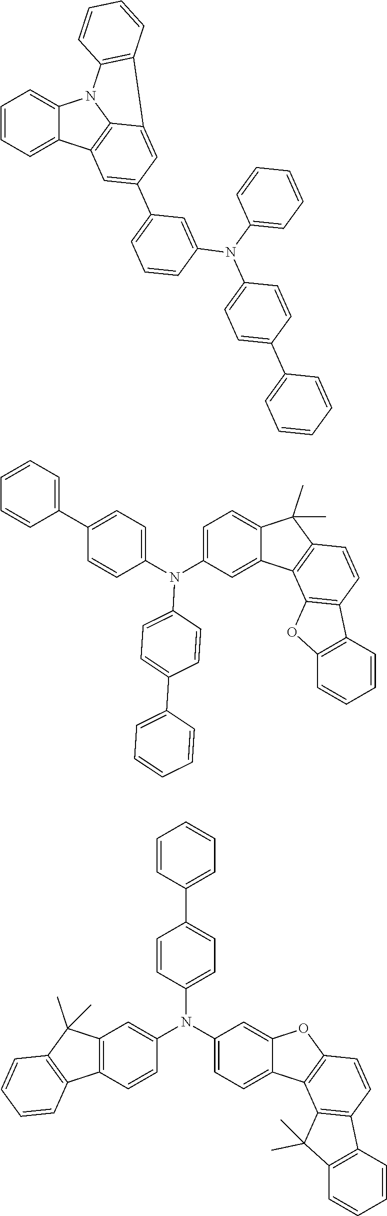 [SMILES: c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21 ; CC1(C)c2ccccc2-c2ccc(N(c3ccc(-c4ccccc4)cc3)c3ccc4c(c3)oc3ccc5c(c34)C(C)(C)c3ccccc3-5)cc21]

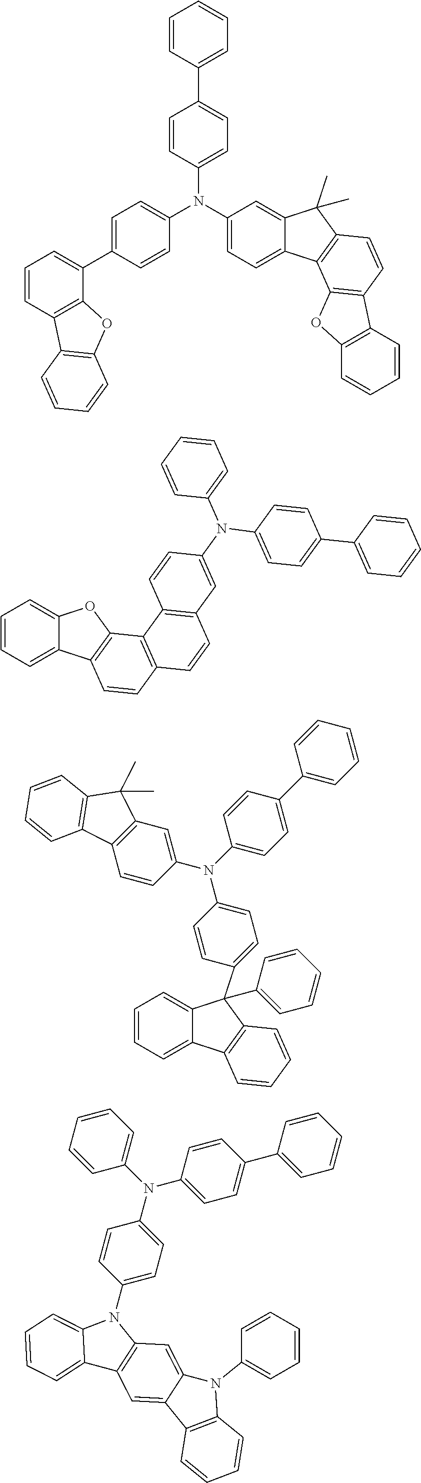

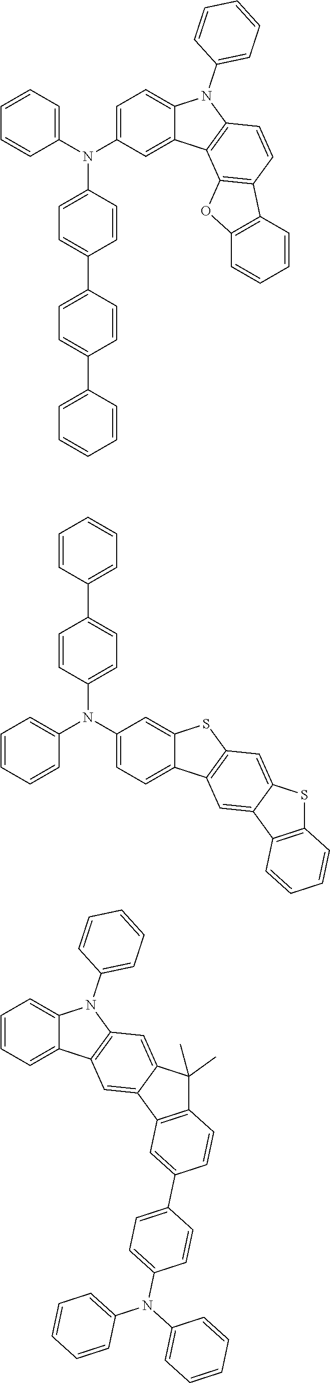 [SMILES: c1ccc(-c2ccc(-c3ccc(N(c4ccccc4)c4ccc5c(c4)c4c6oc7ccccc7c6ccc4n5-c4ccccc4)cc3)cc2)cc1 ; c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21]

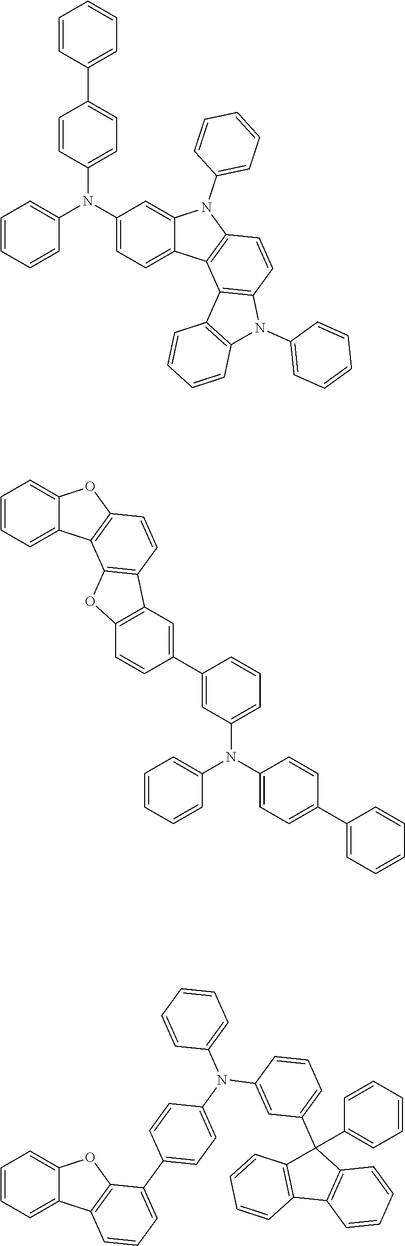 [SMILES: c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1 ; c1ccc(N(c2ccc(-c3cccc4c3oc3ccccc34)cc2)c2cccc(C3(c4ccccc4)c4ccccc4-c4ccccc43)c2)cc1]

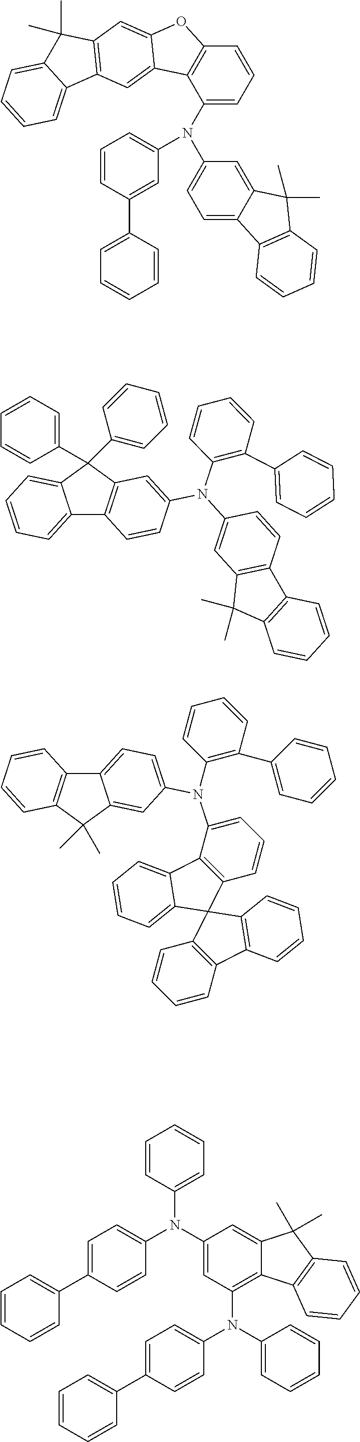 [SMILES: CC1(C)c2ccccc2-c2ccc(N(c3cccc(-c4ccccc4)c3)c3cccc4oc5cc6c(cc5c34)-c3ccccc3C6(C)C)cc21 ; CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccccc3-c3ccccc3)c3cccc4c3-c3ccccc3C43c4ccccc4-c4ccccc43)cc21 ; CC1(C)c2ccccc2-c2c(N(c3ccccc3)c3ccc(-c4ccccc4)cc3)cc(N(c3ccccc3)c3ccc(-c4ccccc4)cc3)cc21]

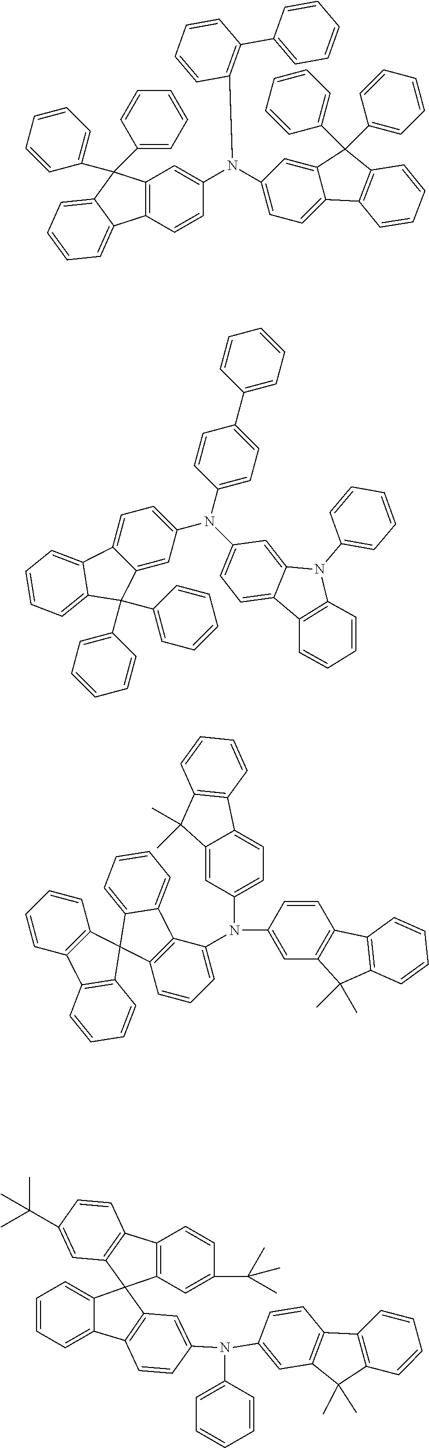 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2]

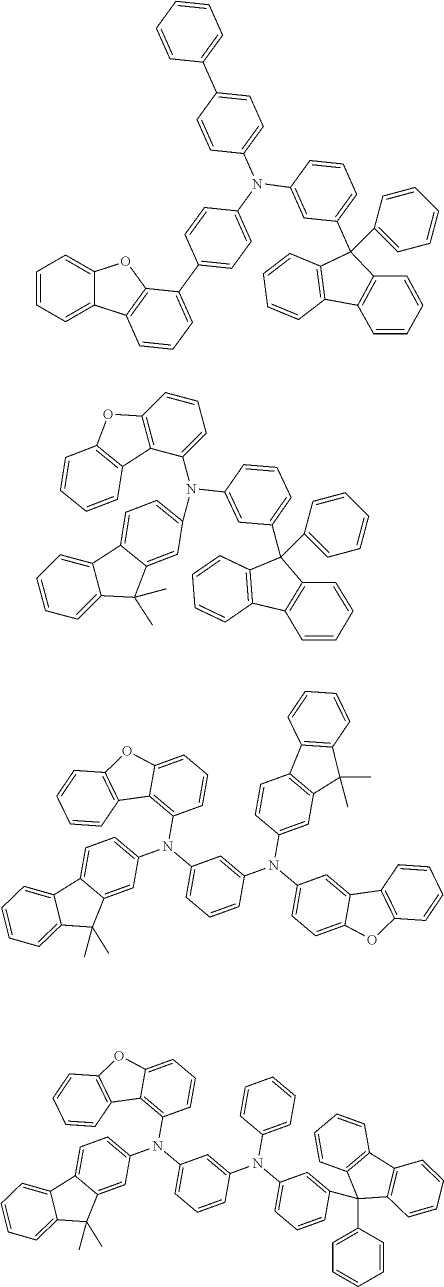 [SMILES: c1ccc(-c2ccc(N(c3ccc(-c4cccc5c4oc4ccccc45)cc3)c3cccc(C4(c5ccccc5)c5ccccc5-c5ccccc54)c3)cc2)cc1 ; CC1(C)c2ccccc2-c2ccc(N(c3cccc(C4(c5ccccc5)c5ccccc5-c5ccccc54)c3)c3cccc4oc5ccccc5c34)cc21 ; CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21 ; CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21]

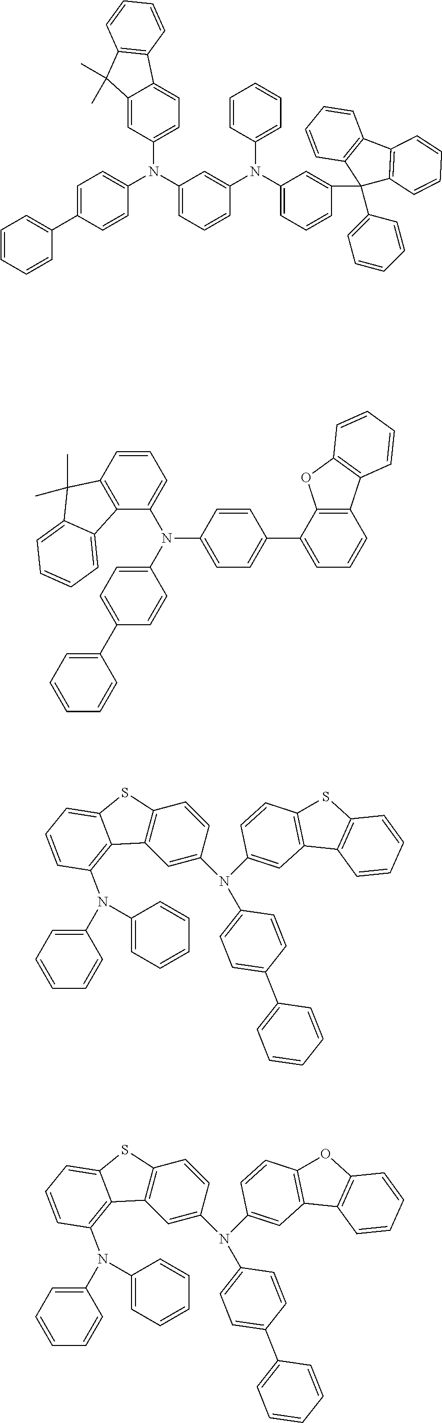 [SMILES: CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21 ; CC1(C)c2ccccc2-c2c(N(c3ccc(-c4ccccc4)cc3)c3ccc(-c4cccc5c4oc4ccccc45)cc3)cccc21 ; c1ccc(-c2ccc(N(c3ccc4sc5ccccc5c4c3)c3ccc4sc5cccc(N(c6ccccc6)c6ccccc6)c5c4c3)cc2)cc1 ; c1ccc(-c2ccc(N(c3ccc4oc5ccccc5c4c3)c3ccc4sc5cccc(N(c6ccccc6)c6ccccc6)c5c4c3)cc2)cc1]

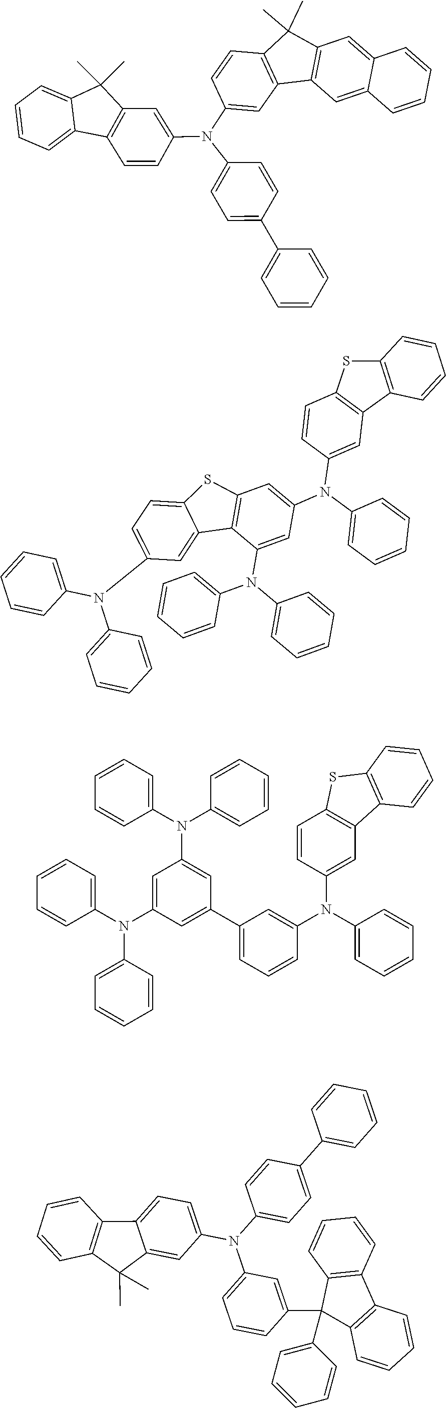 [SMILES: CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

In the hole transport region 140, other suitable compounds may be used in addition to the compound.

In an implementation, the charge transport region may be, e.g., an electron transport region 150.

The electron transport region 150 may help further increase electron injection and/or electron mobility and block holes between the cathode 110 and the light emitting layer 130.

In an implementation, the electron transport region 150 may include an electron transport layer between the cathode 110 and the light emitting layer 130, and an electron transport auxiliary layer between the light emitting layer 130 and the electron transport layer, and a compound of Group C may be included in at least one of the electron transport layer and the electron transport auxiliary layer.

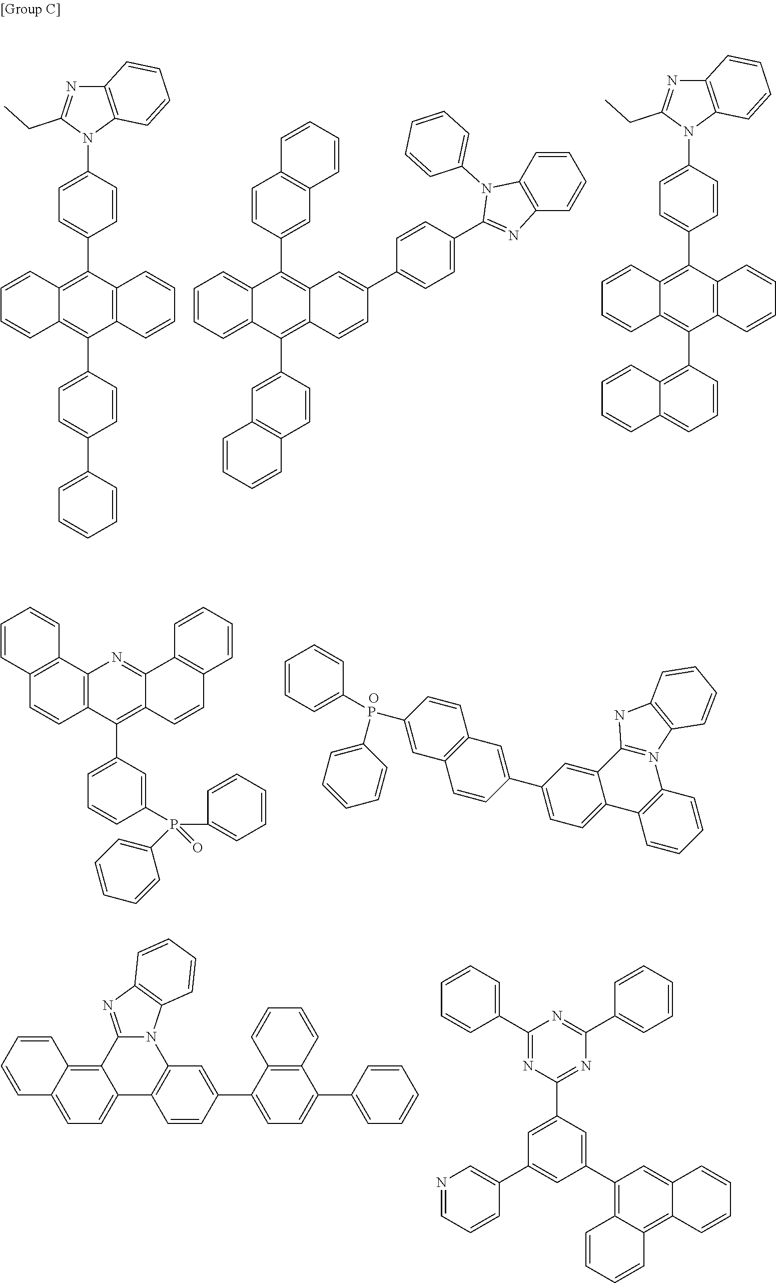

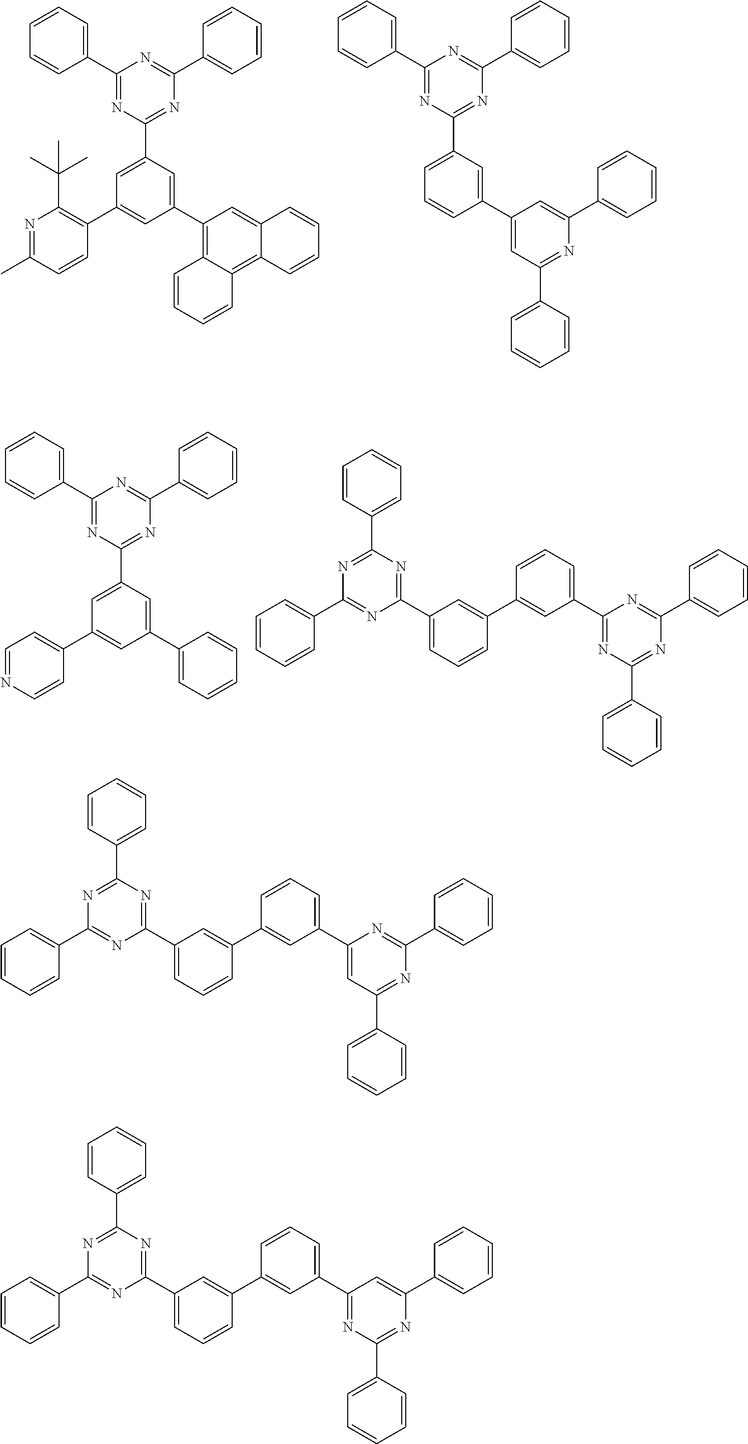

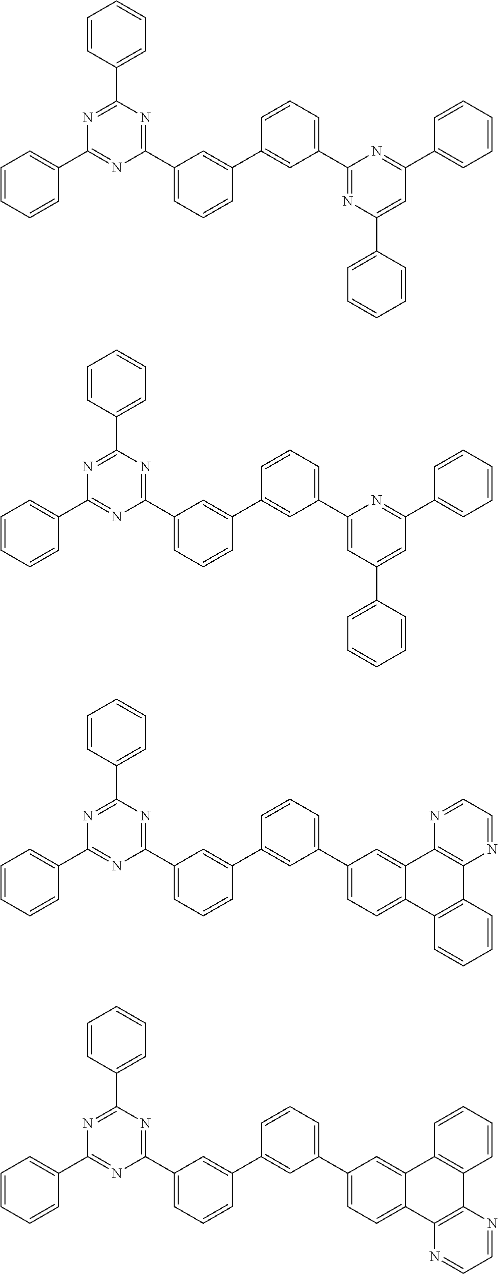 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

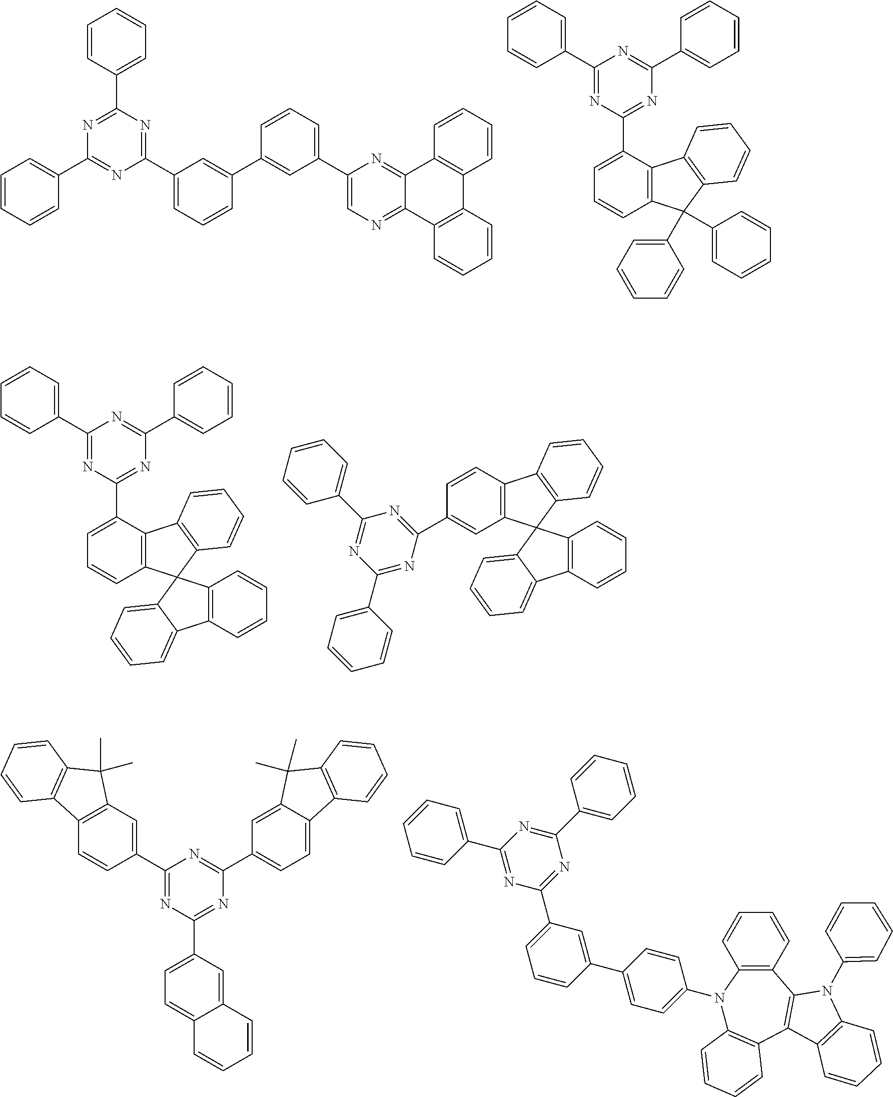

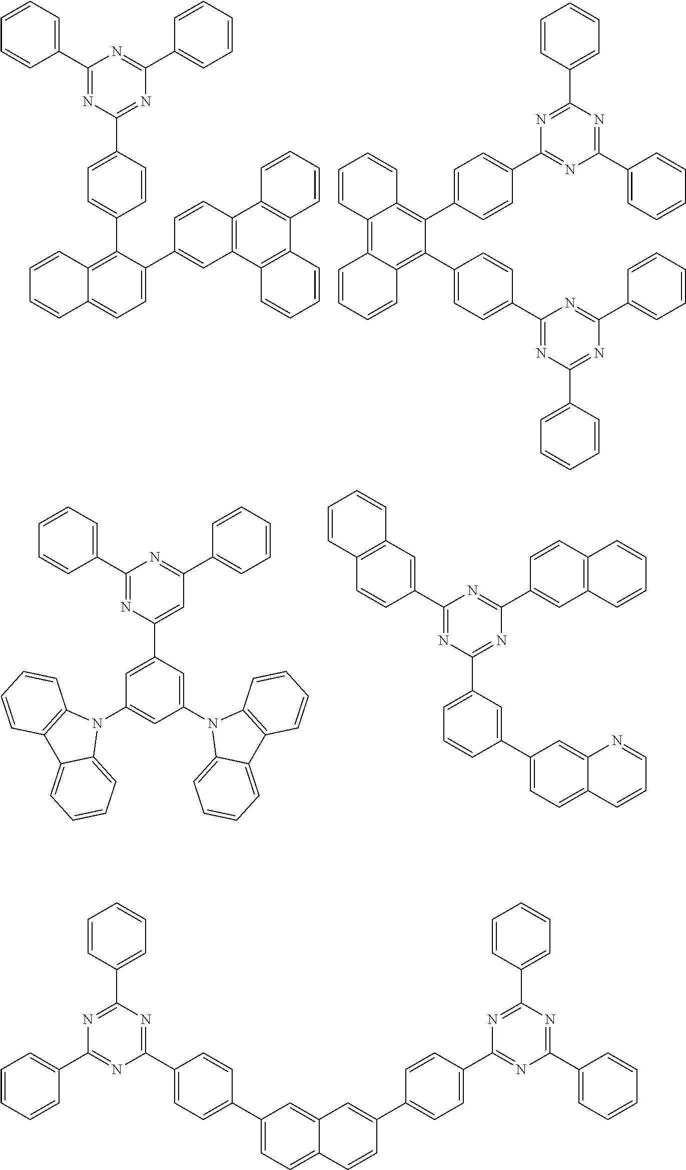

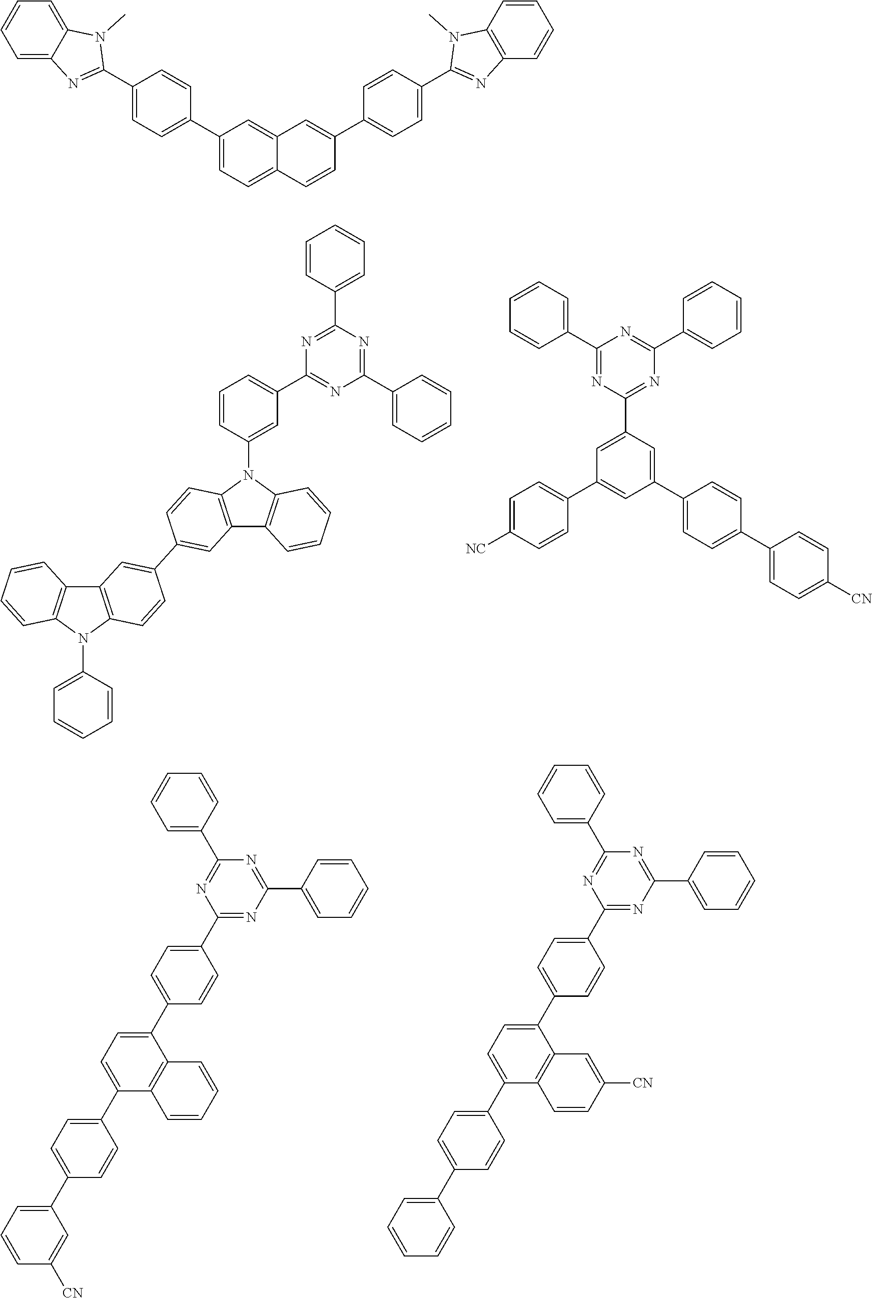 [SMILES: Cn1c(-c2ccc(-c3ccc4ccc(-c5ccc(-c6nc7ccccc7n6C)cc5)cc4c3)cc2)nc2ccccc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

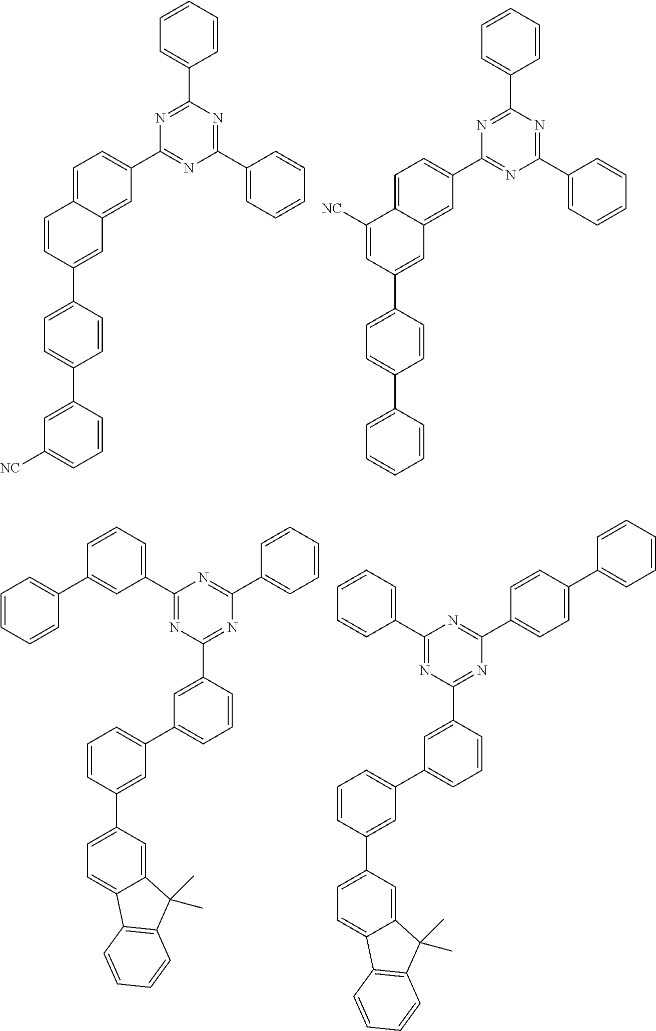

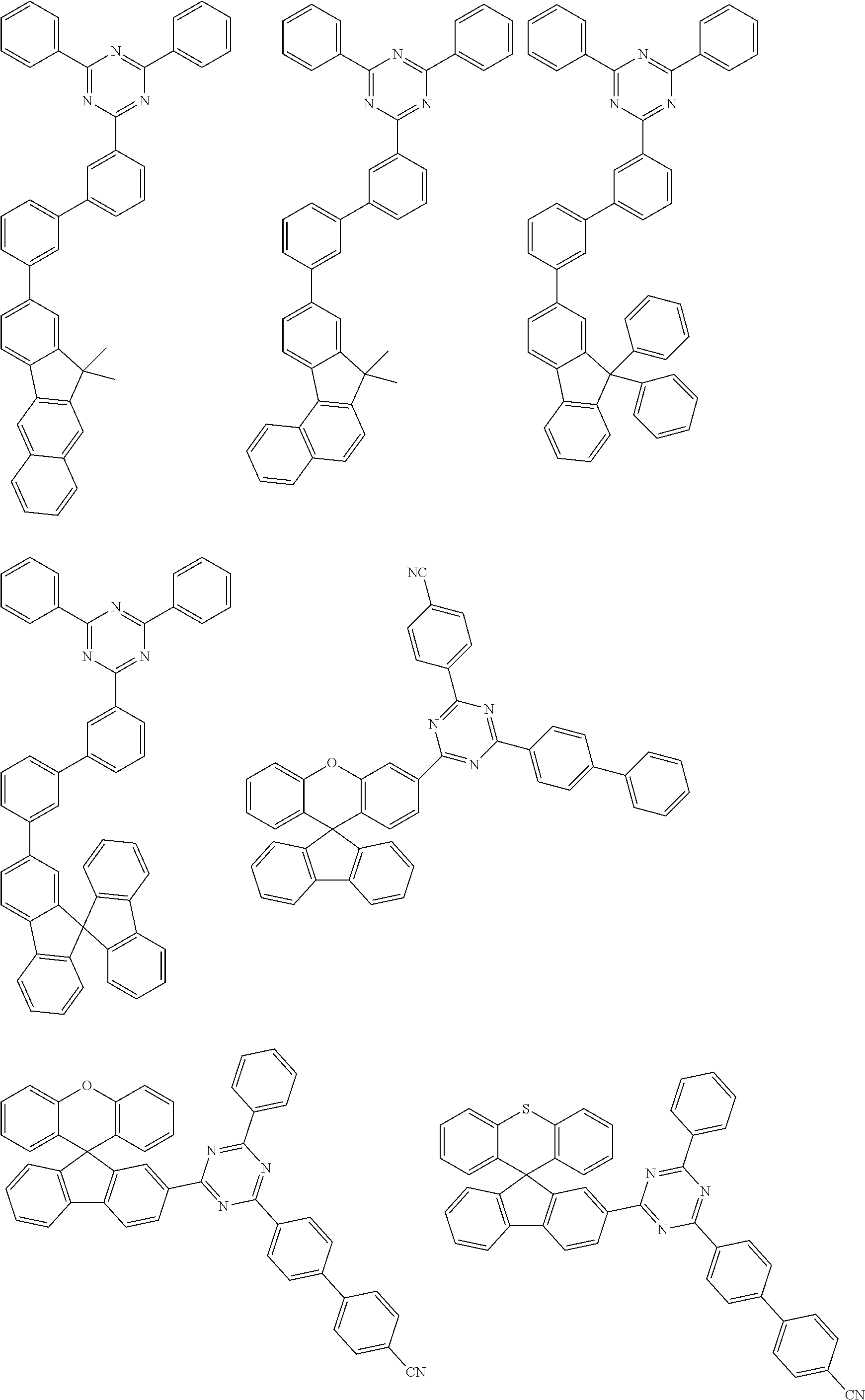

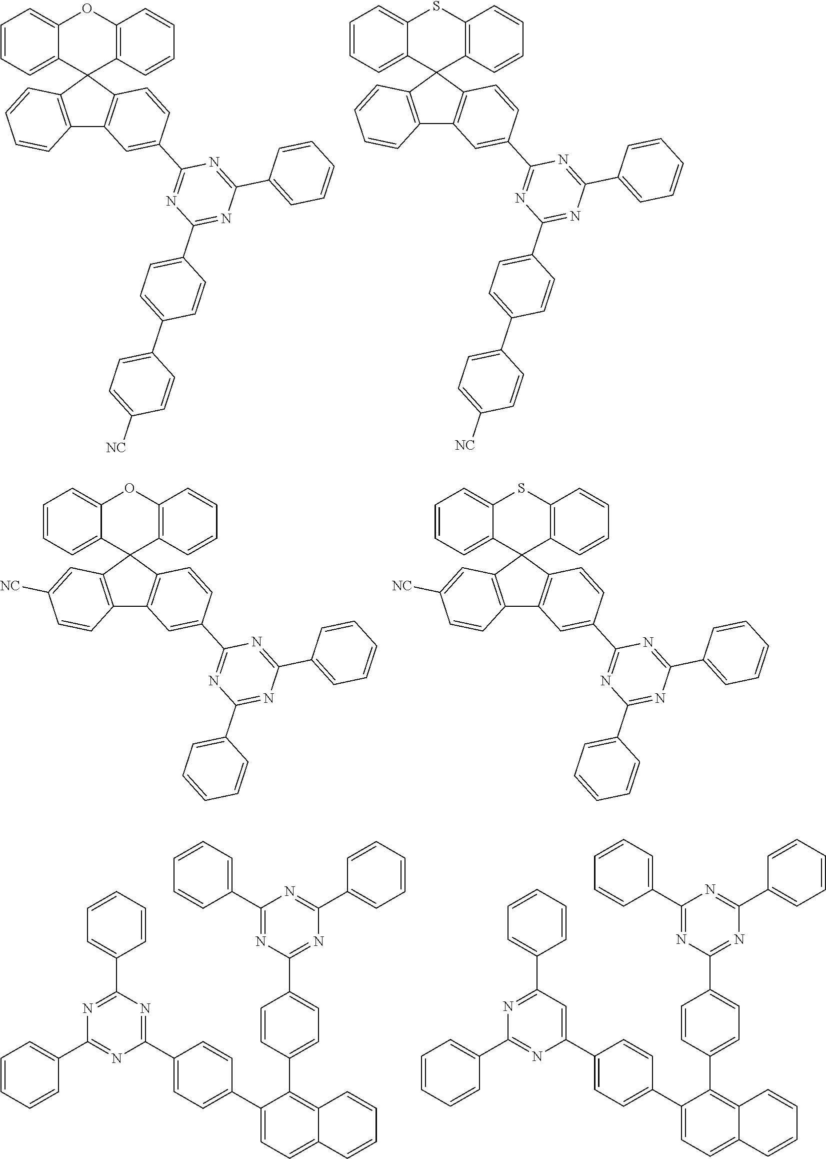

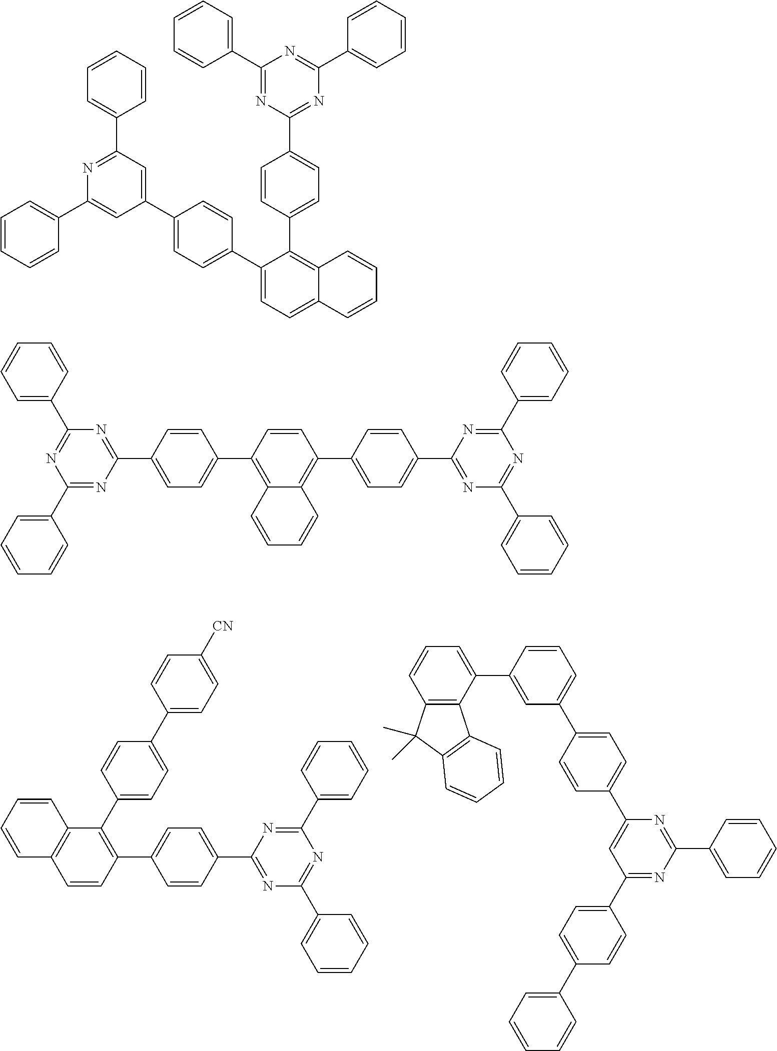

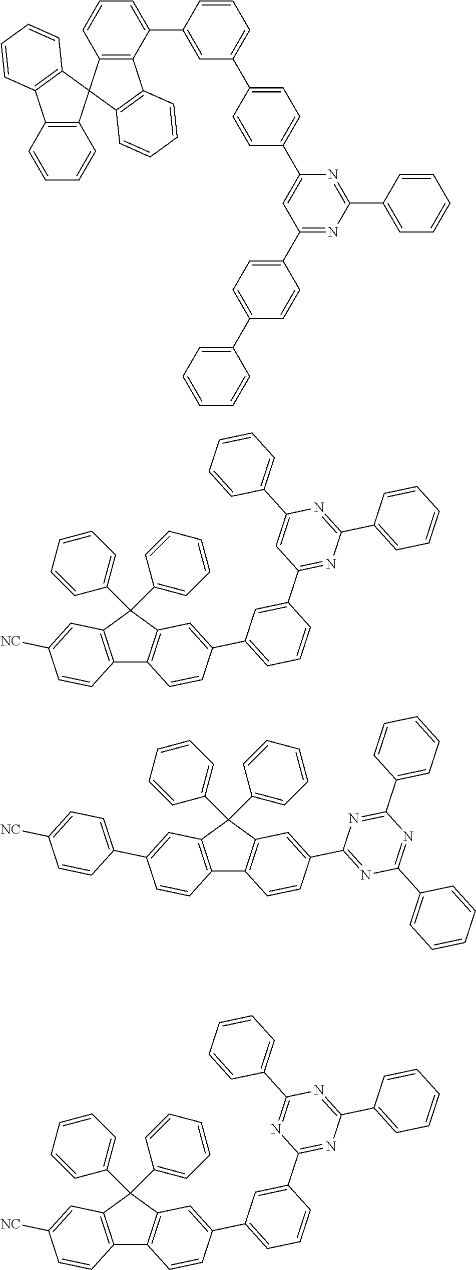

An embodiment may provide an organic light emitting diode including a light emitting layer as an organic layer.

Another embodiment may provide an organic light emitting diode including a light emitting layer and a hole transport region as an organic layer.

Another embodiment may provide an organic light emitting diode including a light emitting layer and an electron transport region as an organic layer.

As shown in the FIGURE, the organic light emitting diode according to an embodiment may include a hole transport region 140 and an electron transport region 150 in addition to the light emitting layer 130 as the organic layer 105.

In an implementation, the organic light emitting diode may further include an electron injection layer, a hole injection layer, or the like, in addition to the light emitting layer as the aforementioned organic layer.

The organic light emitting diode 100 may be produced by forming an anode or a cathode on a substrate, forming an organic layer using a dry film formation method such as a vacuum deposition method (evaporation), sputtering, plasma plating, or ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in examples and synthesis examples were purchased from Sigma-Aldrich Co. Ltd., TCI Inc., Tokyo Chemical Industry, or P&H Tech as far as there in no particular comment or were synthesized by known methods.

(Preparation of Compound for Organic Optoelectronic Device)

The compound was synthesized through the following steps.

### Preparation Synthesis Example 1: Synthesis of Compound A1

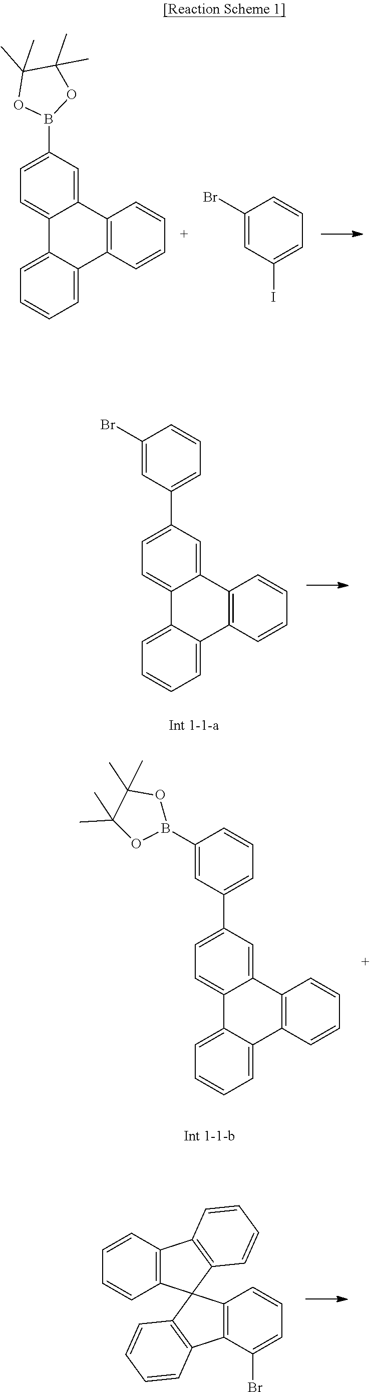

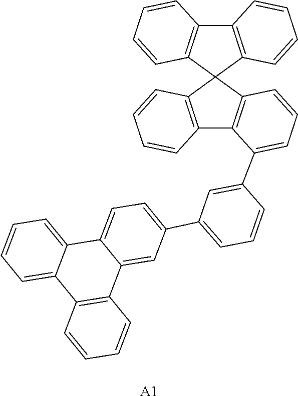 [SMILES: c1cc(-c2ccc3c4ccccc4c4ccccc4c3c2)cc(-c2cccc3c2-c2ccccc2C32c3ccccc3-c3ccccc32)c1 ; "A1"]

1st Step: Synthesis of Intermediate Int 1-1-a

50.3 g (142.1 mmol) of 4,4,5,5-tetramethyl-2-(triphenylen-2-yl)-1,3,2-dioxaborolane, 40.2 g (142.1 mmol) of 1-bromo-3-iodobenzene, 29.5 g (213.2 mmol) of K₂CO₃, and 4.9 g (4.3 mmol) of Pd(PPh₃)₄ were suspended in 280 ml of THF and 110 ml of distilled water and then, stirred under reflux for 8 hours under a nitrogen flow. When a reaction was completed, the resultant was extracted with DCM (dichloromethane) and treated through column chromatography (hexane:DCM (20%)), obtaining 39.3 g (78%) of Intermediate Int 1-1-a as a solid.

2nd Step: Synthesis of Intermediate Int 1-1-b

77 g (203.3 mmol) of Intermediate Int 1-1-a, 59.4 g (233.8 mmol) of bis(pinacolato)diboron, 4.8 g (5.9 mmol) of Pd(dppf)Cl₂, and 28.9 g (294.8 mmol) of potassium acetate were put in a round-bottomed flask and dissolved in 400 ml of DMF. The mixture was stirred under reflux at 120° C. for 12 hours. When a reaction was completed, the mixture was poured into an excess of distilled water and then, stirred for 1 hour. A solid was filtered therefrom and dissolved in DCM. After removing moisture therefrom with MgSO₄, an organic solvent was filtered therefrom with a silica gel pad and removed under a reduced pressure. A solid obtained therefrom was recrystallized with ethyl acetate and hexane, obtaining 41.8 g (70%) of Intermediate Int 1-1-b.

3rd Step: Synthesis of Compound A1

61.2 g (142.1 mmol) of Intermediate Int 1-1-b, 56.2 g (142.1 mmol) of 4-bromo-9,9′-spirobi[9H-fluorene], 29.5 g (213.2 mmol) of K₂CO₃, and 4.9 g (4.3 mmol) of Pd(PPh₃)₄ were suspended in 280 ml of THF and 110 ml of distilled water and then, stirred under reflux for 8 hours for a nitrogen flow. When a reaction was completed, the resultant was extracted with DCM and treated column chromatography (hexane:DCM (20%)), obtaining 39.3 g (78%) of Compound A1 as a solid.

LC-Mass (theoretical value: 618.23 g/mol, measured value: M+=619.40 g/mol)

### Preparation Synthesis Example 2: Synthesis of Compound A2

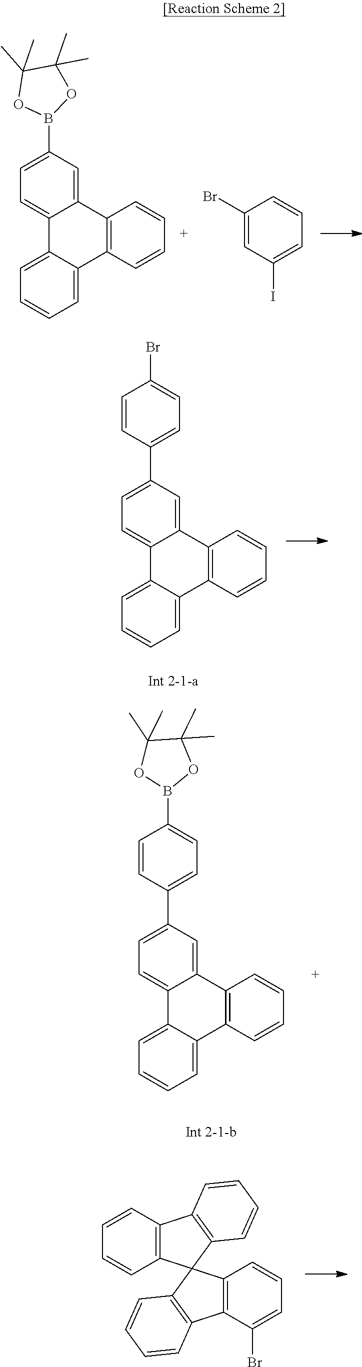

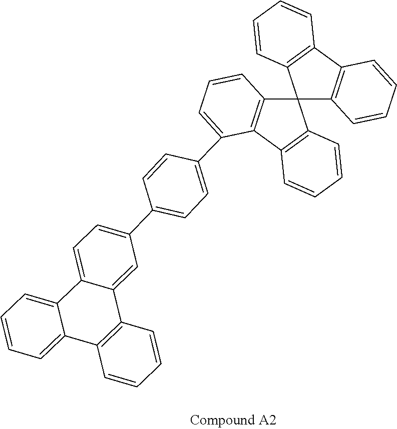 [SMILES: c1ccc2c(c1)-c1ccccc1C21c2ccccc2-c2c(-c3ccc(-c4ccc5c6ccccc6c6ccccc6c5c4)cc3)cccc21]

1st Step: Synthesis of Intermediate Int 2-1-a

50.3 g (142.1 mmol) of 4,4,5,5-tetramethyl-2-(triphenylen-2-yl)-1,3,2-dioxaborolane, 40.2 g (142.1 mmol) of 1-bromo-4-iodobenzene, 29.5 g (213.2 mmol) of K₂CO₃, 4.9 g (4.3 mmol) of Pd(PPh₃)₄ were suspended in 280 ml of THF and 110 ml of distilled water under a nitrogen flow and then, stirred under reflux for 8 hours. When a reaction was completed, the resultant was extracted with DCM and treated through column chromatography (hexane:DCM (20%)), obtaining 43.6 g (80%) of Intermediate Int 2-1-a as a solid.

2nd Step: Synthesis of Intermediate Int 2-1-b

77 g (203.3 mmol) of Intermediate Int 2-1-a, 59.4 g (233.8 mmol) of bis(pinacolato)diboron, 4.8 g (5.9 mmol) of Pd(dppf)Cl₂, and 28.9 g (294.8 mmol) of potassium acetate were put in a round-bottomed flask and dissolved in 400 ml of DMF. The mixture was stirred under reflux at 120° C. for 12 hours. When a reaction was completed, the resultant was poured into an excess of distilled water and then, stirred for 1 hour. A solid was filtered therefrom and dissolved in DCM. After removing moisture with MgSO₄, an organic solvent was filtered therefrom with a silica gel pad and removed under a reduced pressure. The solid was recrystallized with ethyl acetate and hexane, obtaining 65.6 g (75%) of Intermediate Int 2-1-b.

3rd Step: Synthesis of Compound A2

61.2 g (142.1 mmol) of Intermediate Int 2-1-b, 56.2 g (142.1 mmol) of 4-bromo-9,9′-spirobi[9H-fluorene], 29.5 g (213.2 mmol) of K₂CO₃, and 4.9 g (4.3 mmol) of Pd(PPh₃)₄ were suspended in 280 ml of THF and 110 ml of distilled water under a nitrogen flow and then, stirred under reflux for 8 hours. When a reaction was completed, the resultant was extracted with DCM and treated through column chromatography (hexane:DCM (20%)), obtaining 39.3 g (78%) of Compound A2 as a solid.

LC-Mass (theoretical value: 618.23 g/mol, measured value: M+=619.39 g/mol)

### Synthesis Example 1: Synthesis of Compound 1-3

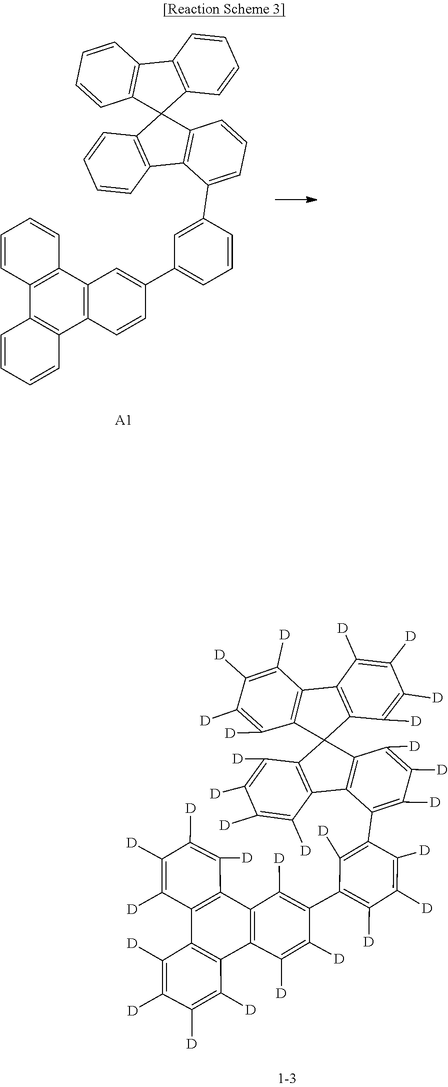

20 g of Compound A1 was put in a round-bottomed flask, and 390 ml of benzene-D₆ was added thereto and then, stirred. 14 ml of triflic acid was added thereto and then, refluxed. After 24 hours, the resultant was cooled to ambient temperature, and D₂O was added thereto and then, stirred for 30 minutes. A solid therefrom was dissolved in an excess of DCM and then, neutralized with a K₃PO₄ aqueous solution. After removing an aqueous layer therefrom, an organic layer was silica gel-filtered to remove a solvent and then, recrystallized, obtaining 13 g of Compound 1-3 (white solid, LC-Mass Mz 648.4, C₄₉D₃₀).

### Synthesis Example 2: Synthesis of Compound 1-4

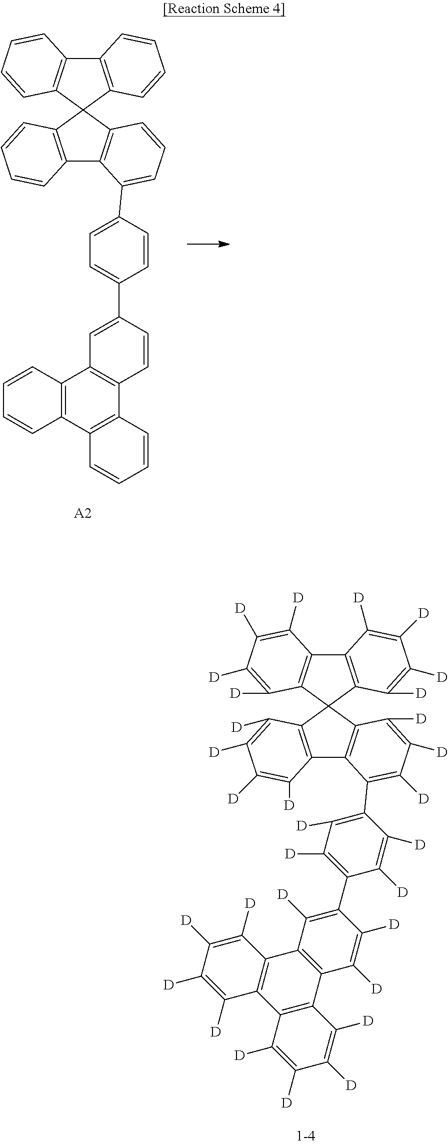

15 g of Compound 1-4 (white solid, LC-Mass Mz 648.4, C₄₉D₃₀) was obtained in the same manner as in Synthesis Example 1 except that 20 g of Compound A2 was used instead of Compound A1.

### Manufacture of Organic Light Emitting Diode

### Example 1

A glass substrate coated with a thin film of indium tin oxide (ITO) was washed with distilled water and ultrasonic waves. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited on the hole transport layer to form a 1,350 Å-thick hole transport layer. On the hole transport layer, Compound B was deposited at a thickness of 350 Å to form a hole transport auxiliary layer. On the hole transport auxiliary layer, 380 Å-thick light emitting layer was formed by using Compound 1-3 obtained in Synthesis Example 1 and doping 10 wt % of PhGD as a dopant by vacuum-deposition. Subsequently, on the light emitting layer, Compound C was deposited at a thickness of 50 Å to form an electron transport auxiliary layer and Compound D and LiQ were simultaneously vacuum-deposited in a weight ratio of 1:1 to form a 300 Å-thick electron transport layer. On the electron transport layer, LiQ and Al were sequentially vacuum-deposited to be 15 Å-thick and 1,200 Å-thick, manufacturing an organic light emitting diode.

ITO/Compound A (3% NDP-9 doping, 100 Å)/Compound A (1,350 Å)/Compound B (350 Å)/EML [90 wt % of a host (Compound 1-3): 10 wt % of PhGD] (380 Å)/Compound C (50 Å)/Compound D:LiQ (300 Å)/LiQ (15 Å)/Al (1,200 Å).
Compound A: N-(biphenyl-4-yl)-9,9-dimethyl-N-(4-(9-phenyl-9H-carbazol-3-yl)phenyl)-9H-fluoren-2-amine
Compound B: N,N-bis(9,9-dimethyl-9H-fluoren-4-yl)-9,9-spirobi(fluorene)-2-amine
Compound C: 2-[3′-(9,9-Dimethyl-9H-fluoren-2-yl)[1,1′-biphenyl]-3-yl]-4,6-diphenyl-1,3,5-triazine
Compound D: 2-[4-[4-(4′-Cyano-1,1′-biphenyl-4-yl)-1-naphthyl]phenyl]-4,6-diphenyl-1,3,5-triazine

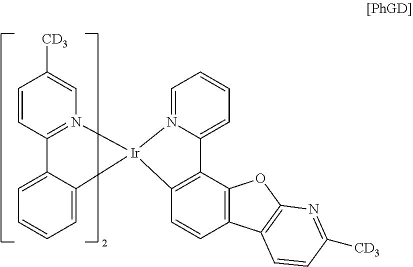

### Comparative Example 1

A diode of Comparative Example 1 was manufactured according to the same manner as Example 1 except that the host was changed as shown in Table 1.

Evaluation: Confirmation of Life-Span Increase Effect

The luminance (cd/m²) was maintained at 6,000 cd/m² and the time for the luminous efficiency (cd/A) to decrease to 96% was measured.

The relative values based on the life-span of Comparative Example 1 are shown in Table 1.

TABLE 1

| | Host | Life-spanT96 (%) |
|---|---|---|
| Example 1 | Compound 1-3 | 121 |
| Comparative Example 1 | Compound A1 | 100 |

Referring to Table 1, the organic light emitting diode according to Example 1 had significantly improved life-span characteristics compared to the organic light emitting diode according to Comparative Example 1.

One or more embodiments may provide a compound for an organic optoelectronic device capable of realizing high-efficiency and long life-span organic optoelectronic device.

A high-efficiency and long life-span organic optoelectronic device may be realized.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by Chemical Formula I: 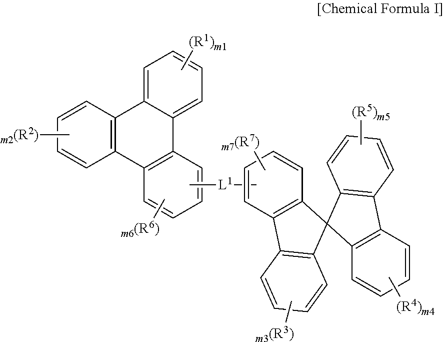
wherein, in Chemical Formula I,
L¹ is a single bond, a substituted or unsubstituted C6 to C20 arylene group, a substituted or unsubstituted C2 to C20 heterocyclic group, or a combination thereof,
R¹ to R⁷ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof,
m1 to m5 are each independently an integer of 1 to 4, and
m6 and m7 are each independently an integer of 1 to 3,
provided that the compound represented by Chemical Formula I satisfies at least one of the following conditions:
L¹ is a C6 to C20 arylene group substituted with at least one deuterium or a C2 to C20 heterocyclic group substituted with at least one deuterium; or
at least one of R¹ to R⁷ is deuterium, a C6 to C30 aryl group substituted with at least one deuterium, or a C2 to C30 heterocyclic group substituted with at least one deuterium.

2. The compound as claimed in claim 1, wherein:
L¹ is a single bond, and
at least one of R¹ to R⁷ is deuterium, a C6 to C30 aryl group substituted with at least one deuterium, or a C2 to C30 heterocyclic group substituted with at least one deuterium.

3. The compound as claimed in claim 1, wherein:
L¹ is a C6 to C20 arylene group substituted with at least one deuterium or a C2 to C20 heterocyclic group substituted with at least one deuterium, and
at least one of R¹ to R⁷ is deuterium, a C6 to C30 aryl group substituted with at least one deuterium, or a C2 to C30 heterocyclic group substituted with at least one deuterium.

4. The compound as claimed in claim 1, wherein L¹ is a phenylene group substituted with at least one deuterium, a biphenylene group substituted with at least one deuterium, a terphenylene group substituted with at least one deuterium, a naphthylene group substituted with at least one deuterium, an anthracenylene group substituted with at least one deuterium, a phenanthrenylene group substituted with at least one deuterium, a triphenylenylene group substituted with at least one deuterium, a fluorenylene group substituted with at least one deuterium, a carbazolylene group substituted with at least one deuterium, a dibenzofuranylene group substituted with at least one deuterium, or a dibenzothiophenylene group substituted with at least one deuterium.

5. The compound as claimed in claim 1, wherein at least one of R¹ to R⁷ is deuterium, a phenyl group substituted with at least one deuterium, a biphenyl group substituted with at least one deuterium, a terphenyl group substituted with at least one deuterium, a naphtyl group substituted with at least one deuterium, an anthracenyl group substituted with at least one deuterium, a phenanthrenyl group substituted with at least one deuterium, a triphenylene group substituted with at least one deuterium, a fluorenyl group substituted with at least one deuterium, a carbazolyl group substituted with at least one deuterium, a dibenzofuranyl group substituted with at least one deuterium, or a dibenzothiophenyl group substituted with at least one deuterium.

6. The compound as claimed in claim 1, wherein:
the compound represented by Chemical Formula I is represented by one of Chemical Formula I-1 to Chemical Formula I-4: 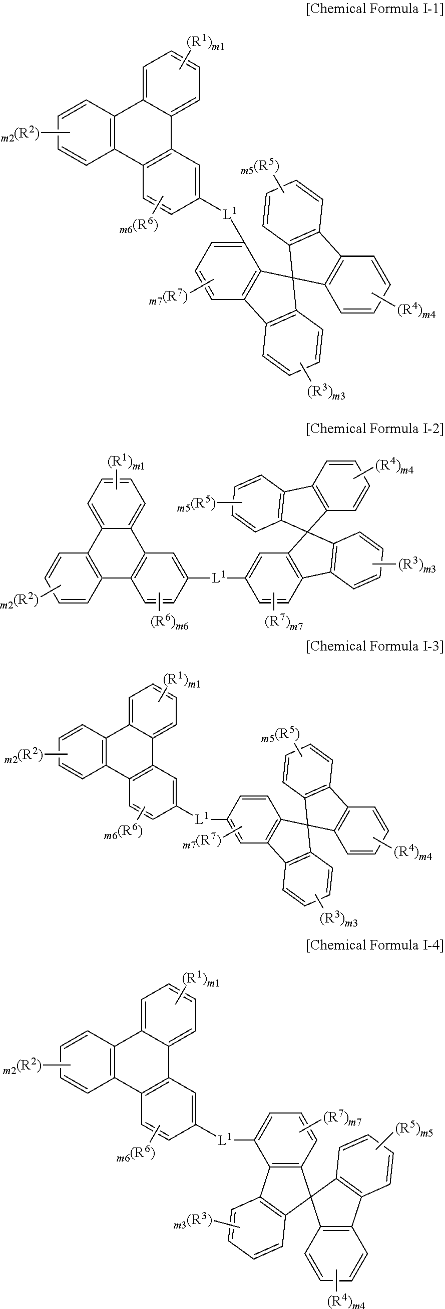
in Chemical Formula I-1 to Chemical Formula I-4, L¹, R¹ to R⁷ and m1 to m7 are defined the same as those of Chemical Formula I.

7. The compound as claimed in claim 1, wherein the compound represented by Chemical Formula I is a compound of Group 1:

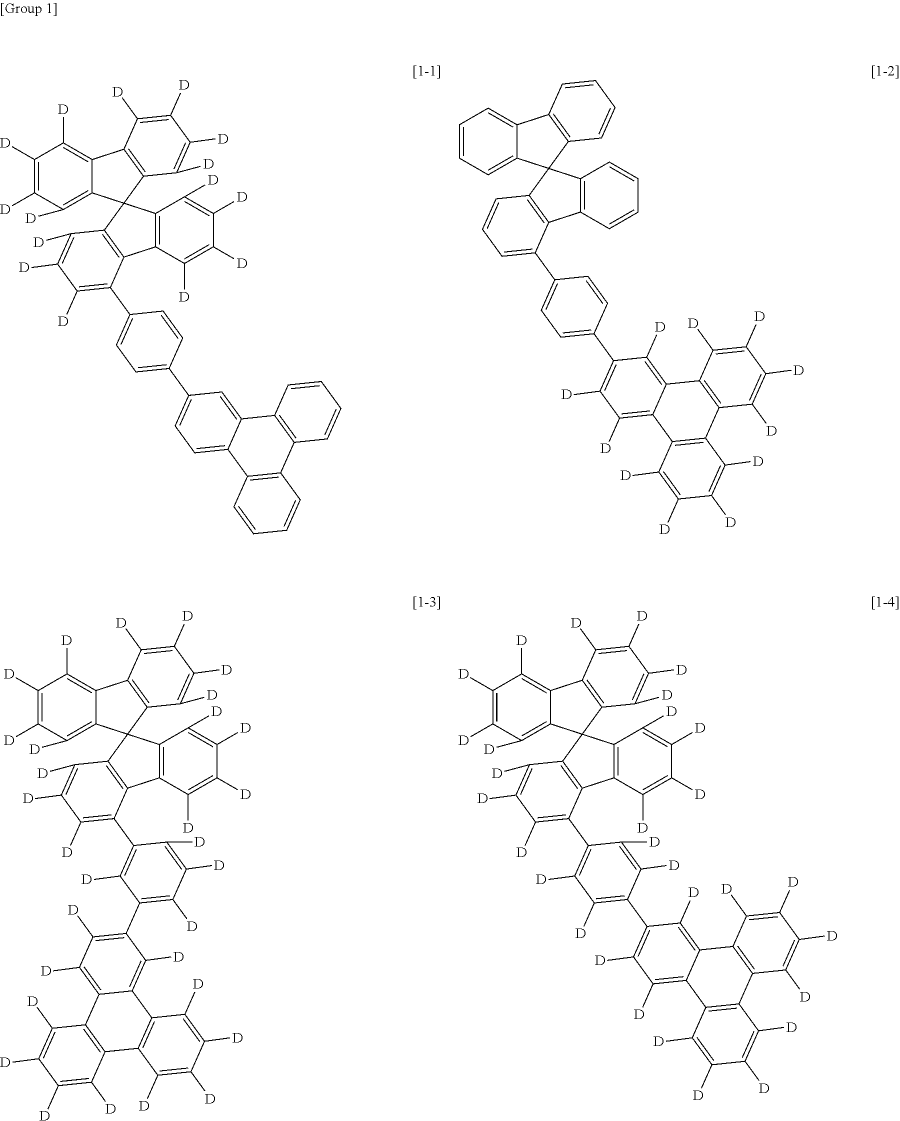

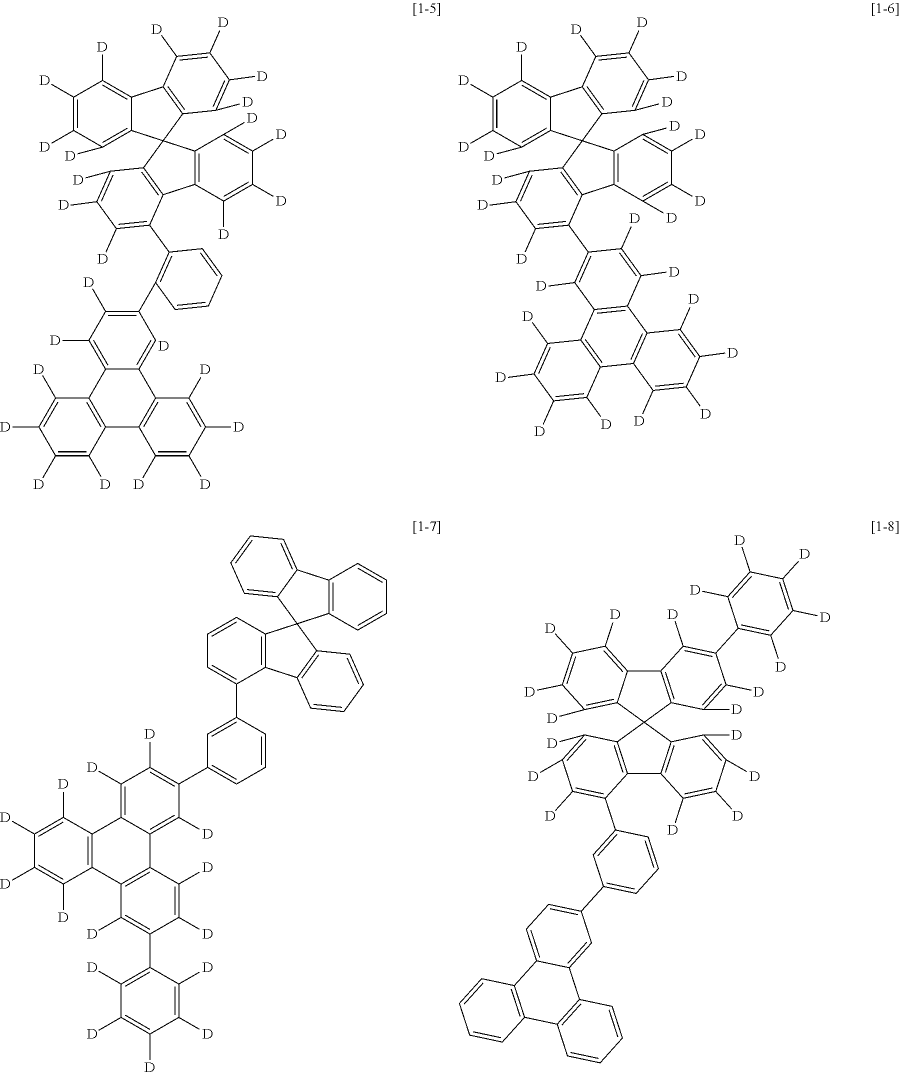

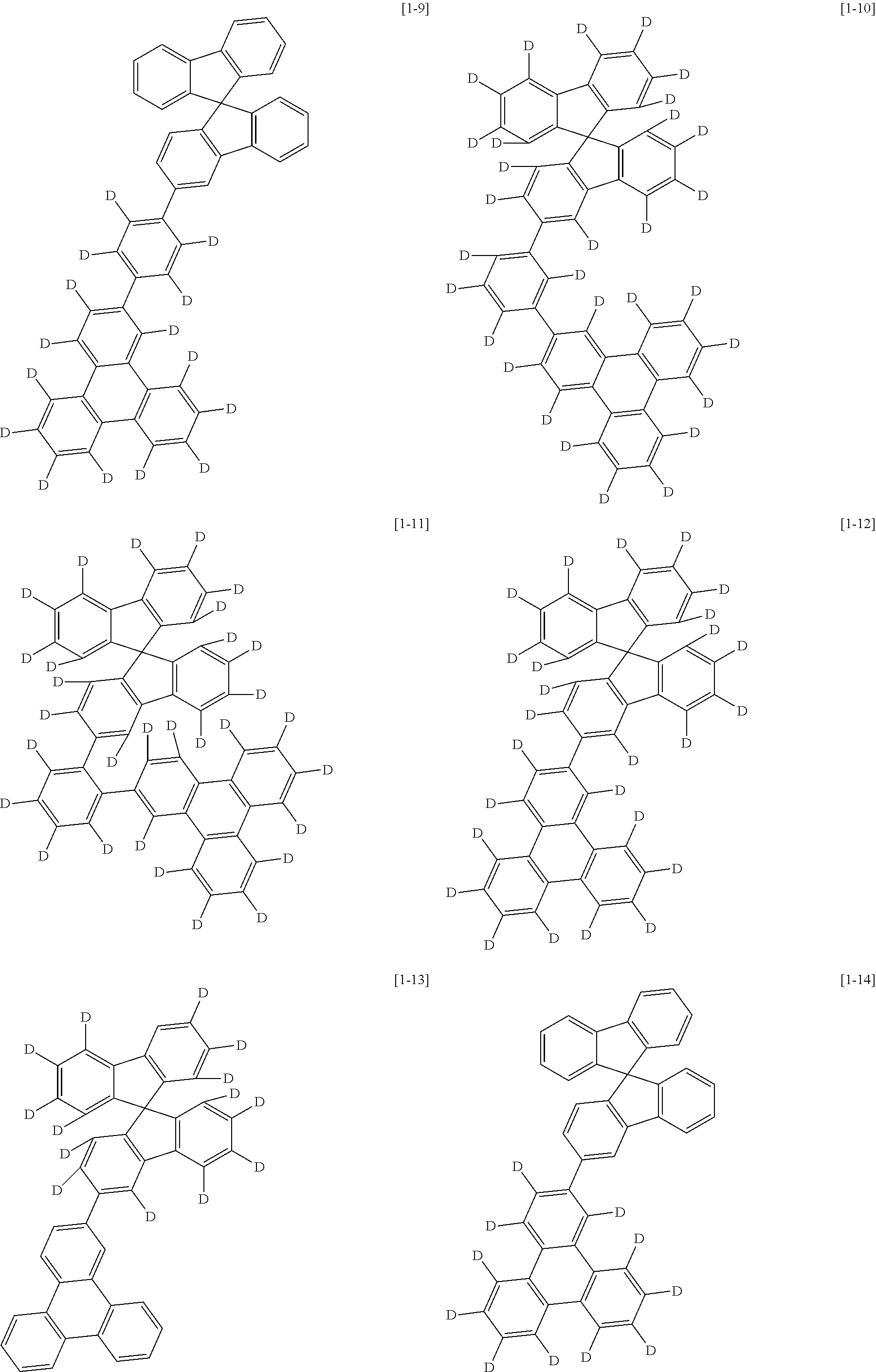

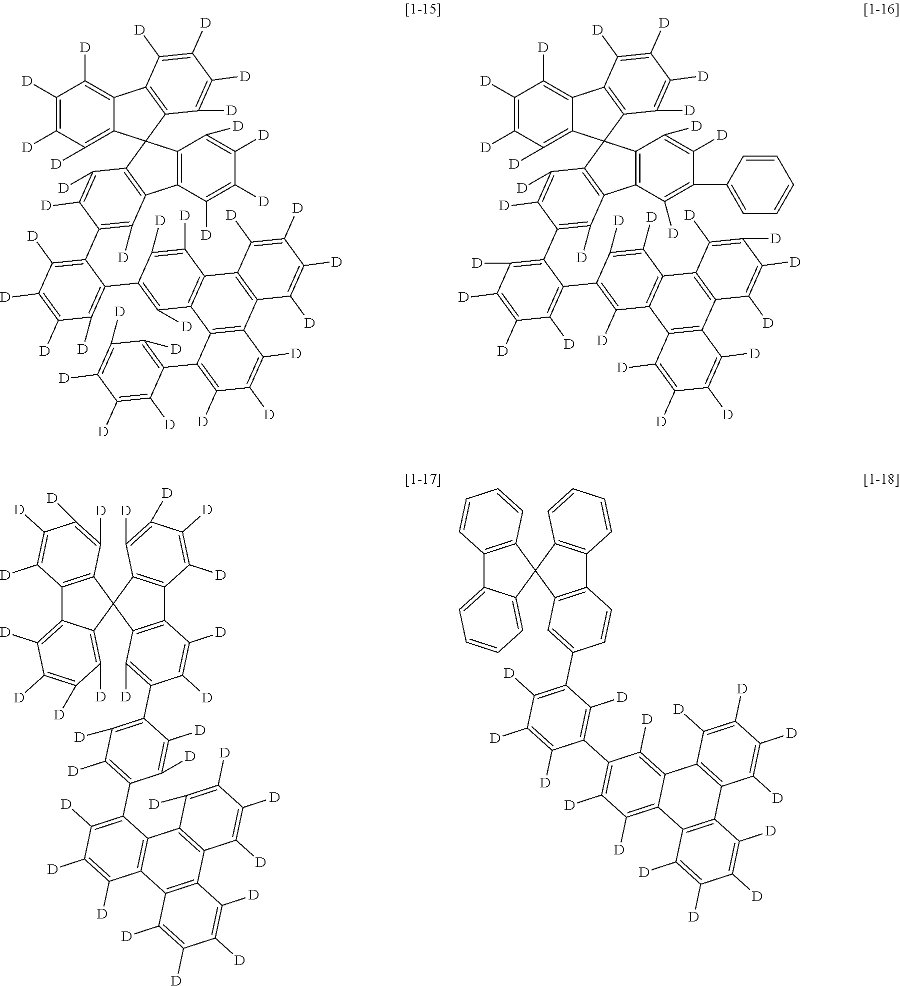

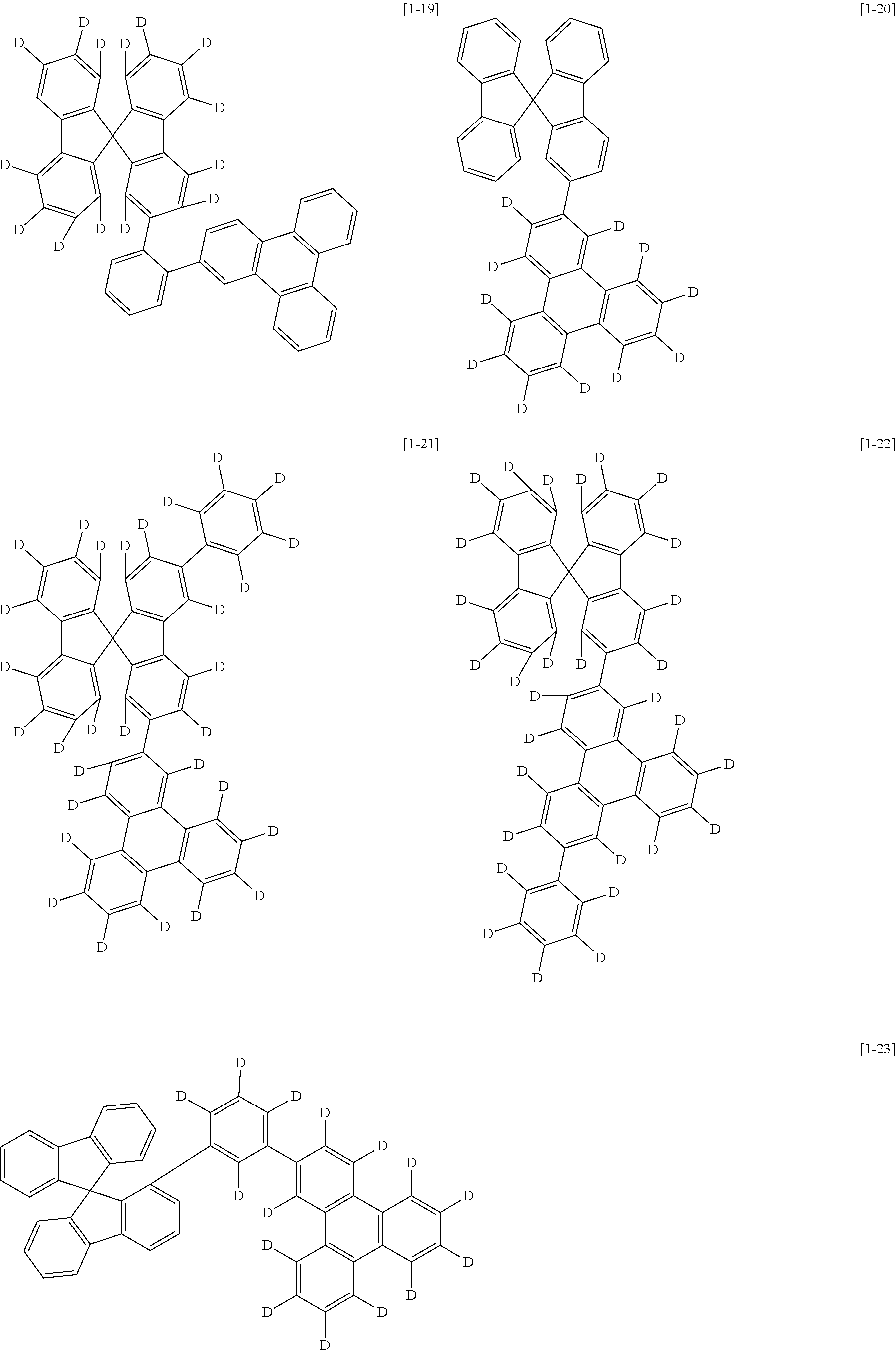

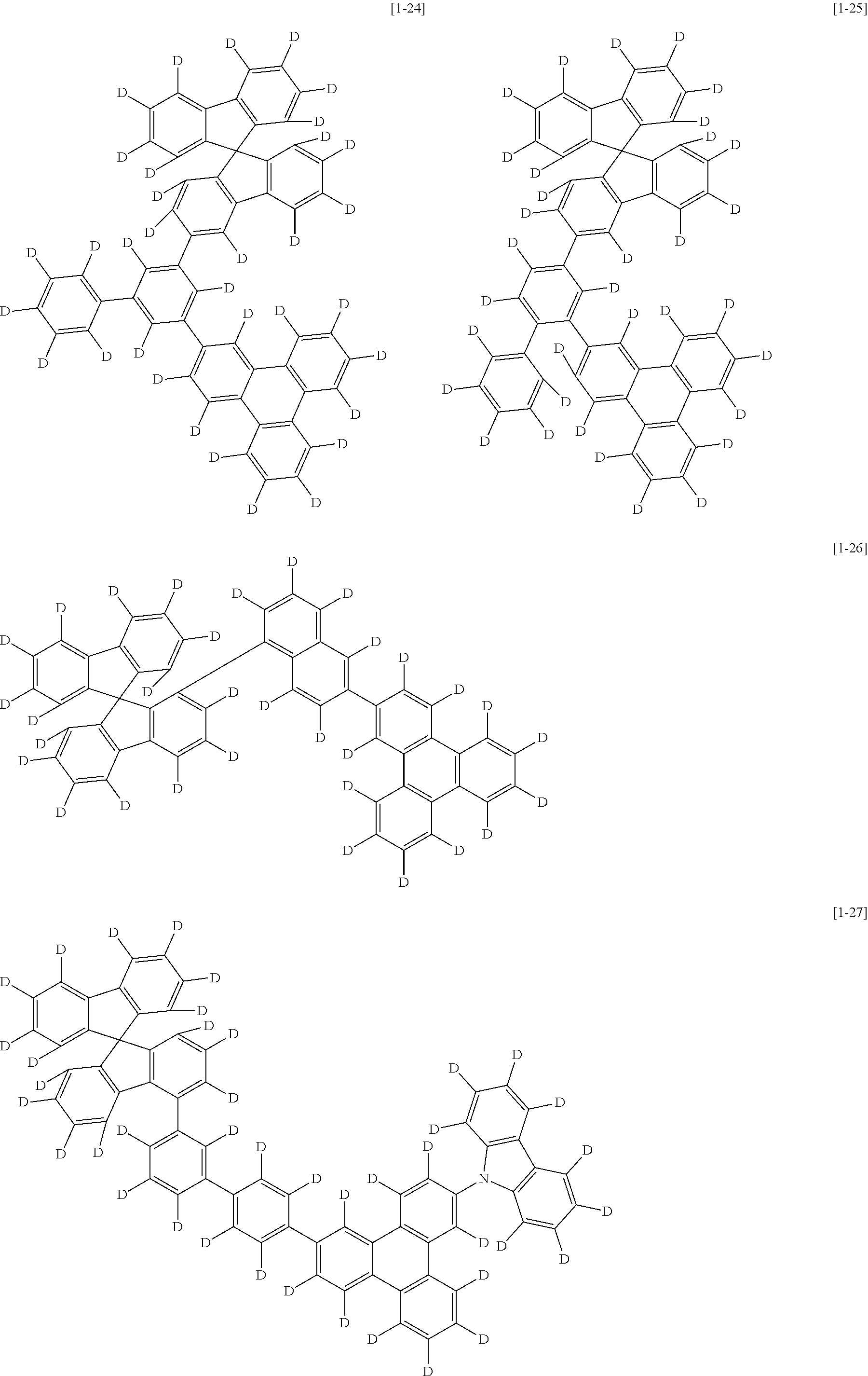

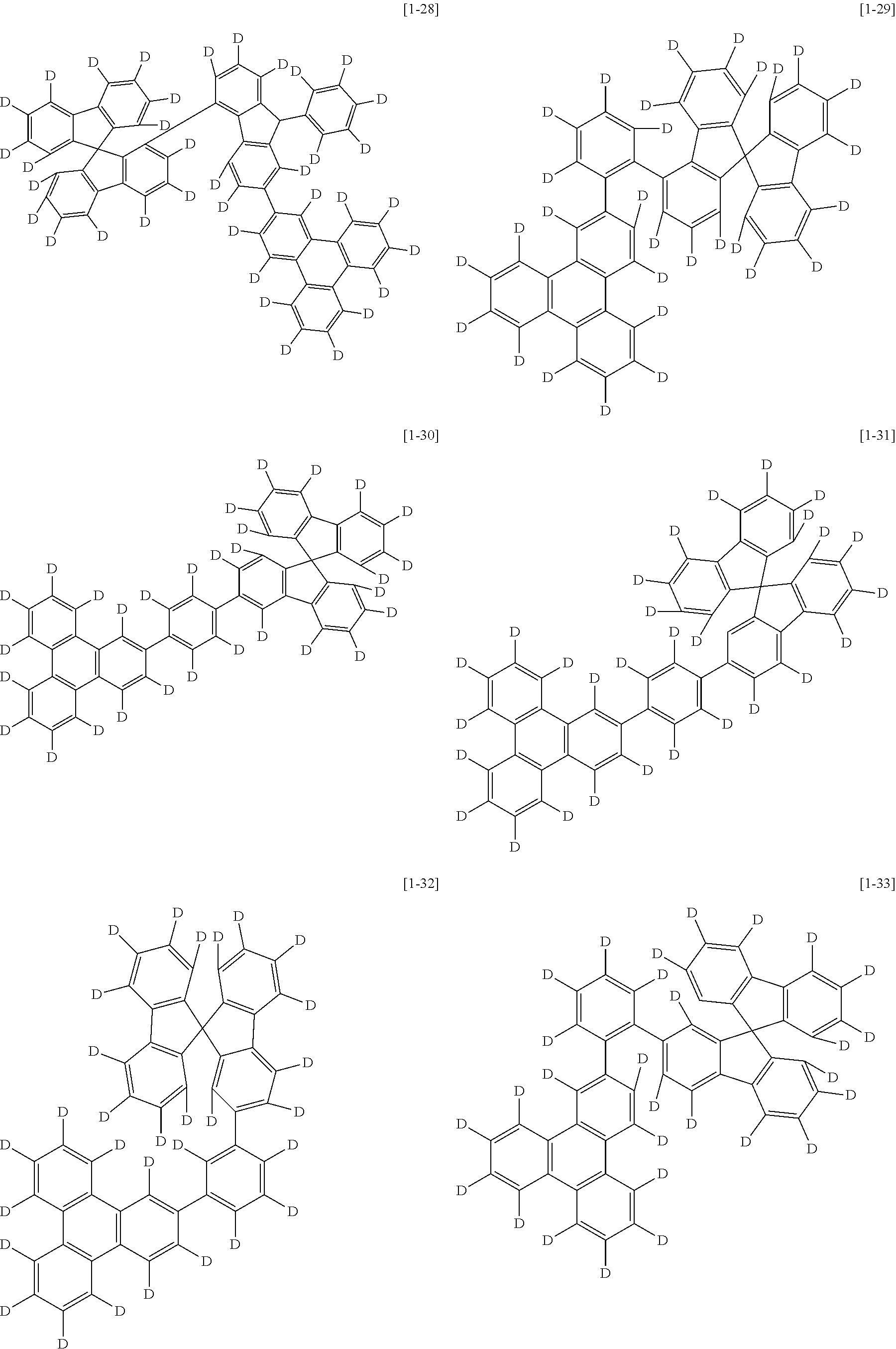

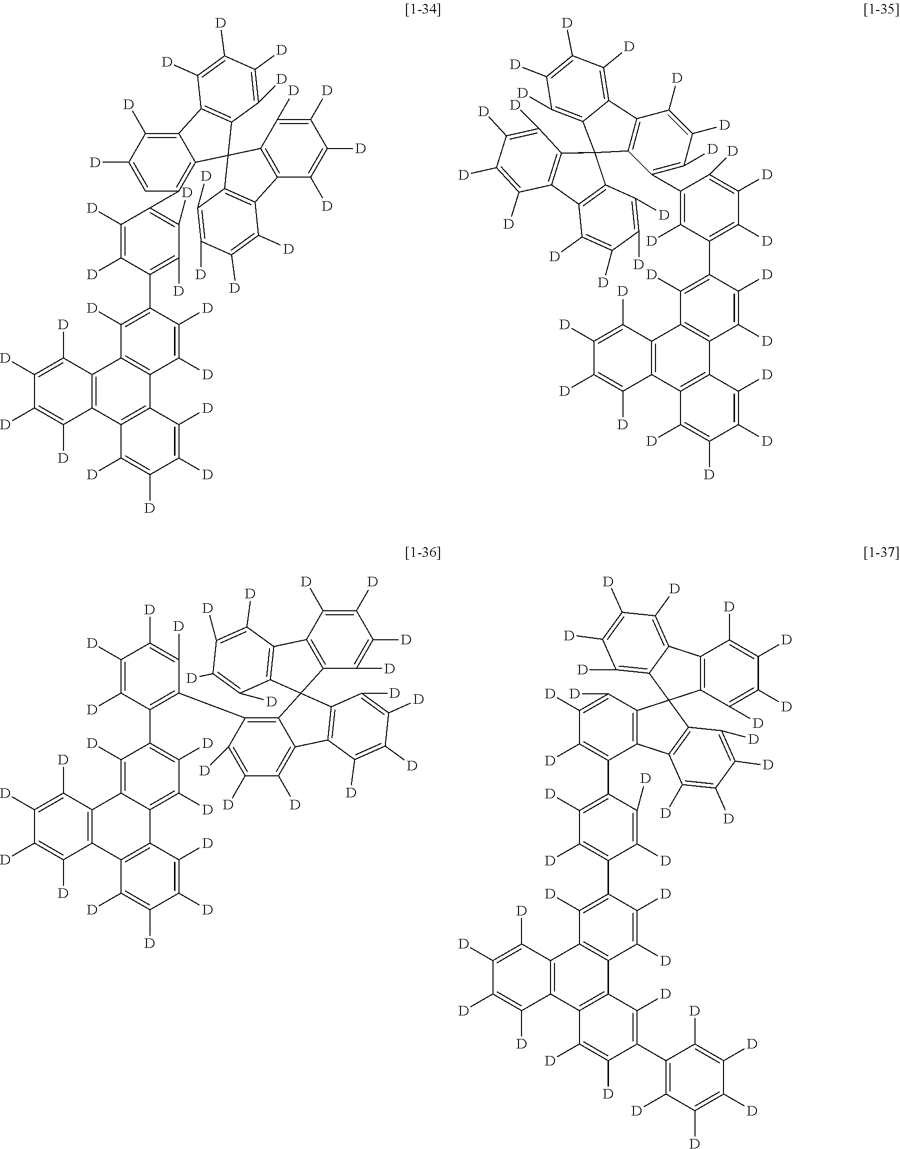

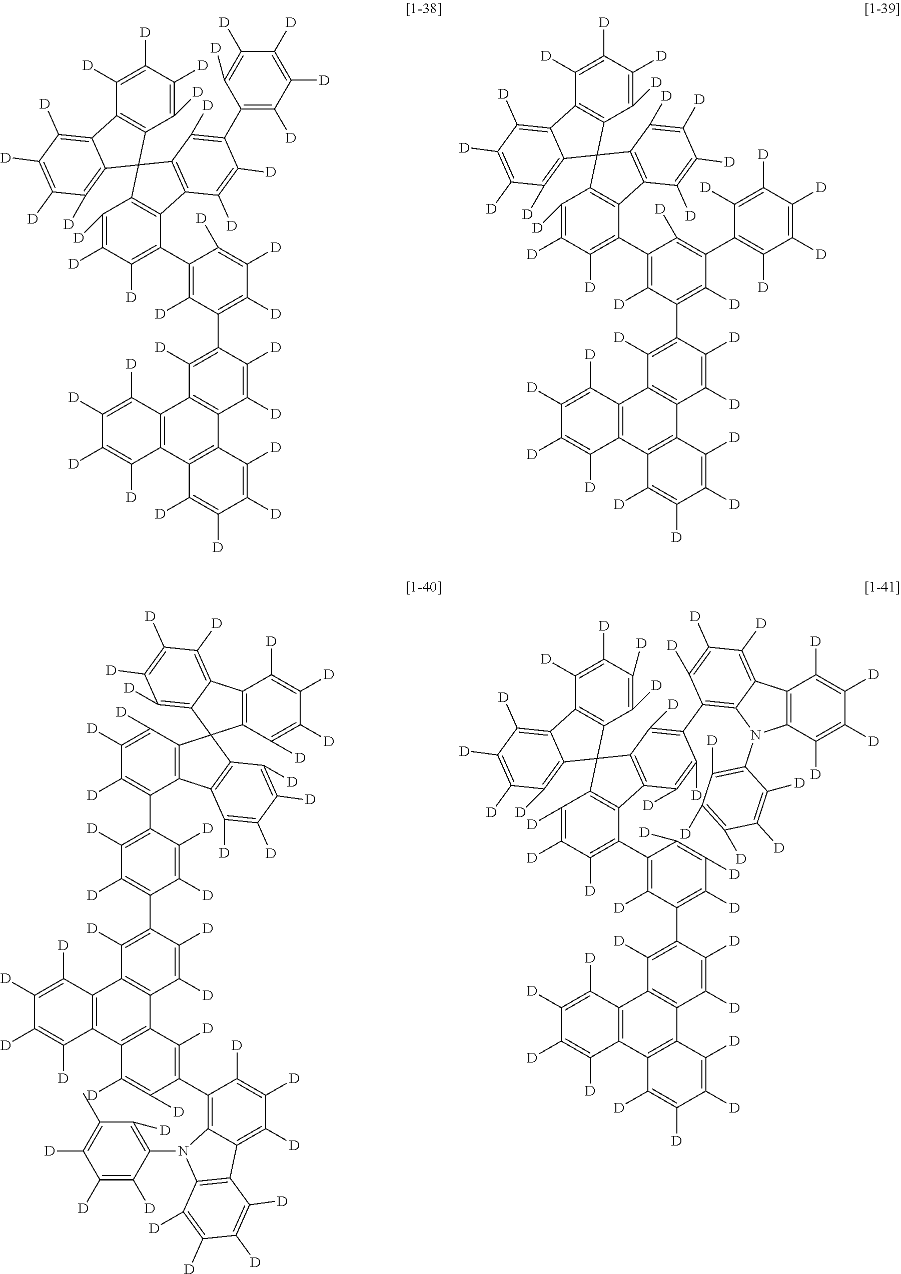

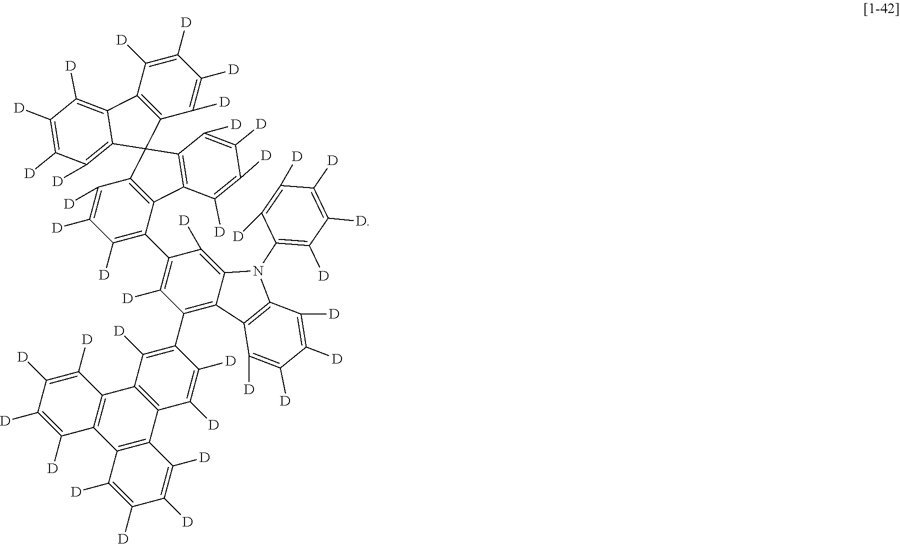

8. An organic optoelectronic device, comprising:
an anode and a cathode facing each other, and
at least one organic layer between the anode and the cathode,
wherein the at least one organic layer includes the compound for an organic optoelectronic device as claimed in claim 1.

9. The organic optoelectronic device as claimed in claim 8, wherein:
the at least one organic layer includes a light emitting layer, and
the light emitting layer includes the compound for an organic optoelectronic device.

10. A display device comprising the organic optoelectronic device as claimed in claim 8.
